SEQUENCE LISTING (86 sequences)

SEQ ID NO: 1 - DNA, 29 bp, Artificial Sequence
  Description of Artificial Sequence Synthetic primer
catcacaaagaagtttctgagaatgcttc

SEQ ID NO: 2 - DNA, 29 bp, Artificial Sequence
  Description of Artificial Sequence Synthetic primer
tgcattcaactcacagagttgaaccttcc

SEQ ID NO: 3 - DNA, 19 bp, Artificial Sequence
  Description of Artificial Sequence Synthetic primer
cacaacaagagctcccatt

SEQ ID NO: 4 - DNA, 20 bp, Artificial Sequence
  Description of Artificial Sequence Synthetic primer
actggggtccttccatctct

SEQ ID NO: 5 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 16 aa, Artificial Sequence
  Description of Artificial Sequence Synthetic peptide
EFPKPSTPPGSSGGAP

SEQ ID NO: 8 - DNA, 21 bp, Artificial Sequence
  Description of Artificial Sequence Synthetic oligonucleotide
aatgcggcggtggtgacagta

SEQ ID NO: 9 - DNA, 21 bp, Artificial Sequence
  Description of Artificial Sequence Synthetic oligonucleotide
aagctcagcacacagaaagac

SEQ ID NO: 10 - DNA, 21 bp, Artificial Sequence
  Description of Artificial Sequence Synthetic oligonucleotide
uaaaaucuuccugcccacctt

SEQ ID NO: 11 - DNA, 21 bp, Artificial Sequence
  Description of Artificial Sequence Synthetic oligonucleotide
ggaagcuguuggcugaaaatt

SEQ ID NO: 12 - RNA, 21 nt, Artificial Sequence
  Description of Artificial Sequence Synthetic oligonucleotide
aagaccagccucuuugcccag

SEQ ID NO: 13 - RNA, 19 nt, Artificial Sequence
  Description of Artificial Sequence Synthetic oligonucleotide
ggaccaggcagaaaacgag

SEQ ID NO: 14 - RNA, 17 nt, Artificial Sequence
  Description of Artificial Sequence Synthetic oligonucleotide
cuaucaggaugacgcgg

SEQ ID NO: 15 - RNA, 21 nt, Artificial Sequence
  Description of Artificial Sequence Synthetic oligonucleotide
ugacacaggcaggcuugacuu

SEQ ID NO: 16 - DNA, 19 bp, Artificial Sequence
  Description of Artificial Sequence Synthetic oligonucleotide
ggtgaagaagggcgtccaa

SEQ ID NO: 17 - DNA, 60 bp, Artificial Sequence
  Description of Artificial Sequence Synthetic oligonucleotide
gatccgttggagctgttggcgtagttcaagagactcgccaacagctccaacttttggaaa

SEQ ID NO: 18 - DNA, 20 bp, Artificial Sequence
  Description of Artificial Sequence Synthetic oligonucleotide
aggtggtgttaacagcagag

SEQ ID NO: 19 - DNA, 21 bp, Artificial Sequence
  Description of Artificial Sequence Synthetic oligonucleotide
aaggtggagcaagcggtggag

SEQ ID NO: 20 - DNA, 21 bp, Artificial Sequence
  Description of Artificial Sequence Synthetic oligonucleotide
aaggagttgaaggccgacaaa

SEQ ID NO: 21 - DNA, 21 bp, Artificial Sequence
  Description of Artificial Sequence Synthetic oligonucleotide
uauggagcugcagaggaugtt

SEQ ID NO: 22 - DNA, 49 bp, Artificial Sequence
  Description of Artificial Sequence Synthetic oligonucleotide
tttgaatatctgtgctgagaacacagttctcagcacagatattcttttt

SEQ ID NO: 23 - DNA, 29 bp, Artificial Sequence
  Description of Artificial Sequence Synthetic oligonucleotide
aatgagaaaagcaaaaggtgccctgtctc

SEQ ID NO: 24 - RNA, 21 nt, Artificial Sequence
  Description of Artificial Sequence Synthetic oligonucleotide
aaucaucaucaagaaagggca

SEQ ID NO: 25 - DNA, 21 bp, Artificial Sequence
  Description of Artificial Sequence Synthetic oligonucleotide
augacugucaggauguugctt

SEQ ID NO: 26 - RNA, 21 nt, Artificial Sequence
  Description of Artificial Sequence Synthetic oligonucleotide
gaacgaauccugaagacaucu

SEQ ID NO: 27 - DNA, 29 bp, Artificial Sequence
  Description of Artificial Sequence Synthetic oligonucleotide
aagcctggctacagcaatatgcctgtctc

SEQ ID NO: 28 - DNA, 21 bp, Artificial Sequence
  Description of Artificial Sequence Synthetic oligonucleotide
ugaccaucaccgaguuuautt

SEQ ID NO: 29 - DNA, 21 bp, Artificial Sequence
  Description of Artificial Sequence Synthetic oligonucleotide
aagtcggacgcaacagagaaa

SEQ ID NO: 30 - DNA, 21 bp, Artificial Sequence
  Description of Artificial Sequence Synthetic oligonucleotide
cuaccuuucuacggacgugtt

SEQ ID NO: 31 - DNA, 21 bp, Artificial Sequence
  Description of Artificial Sequence Synthetic oligonucleotide
ctgcctaaggcggatttgaat

SEQ ID NO: 32 - DNA, 21 bp, Artificial Sequence
  Description of Artificial Sequence Synthetic oligonucleotide
ttauuccuucuucgggaaguc

SEQ ID NO: 33 - DNA, 21 bp, Artificial Sequence
  Description of Artificial Sequence Synthetic oligonucleotide
aaccttctggaacccgcccac

SEQ ID NO: 34 - DNA, 19 bp, Artificial Sequence
  Description of Artificial Sequence Synthetic oligonucleotide
gagcatcttcgagcaagaa

SEQ ID NO: 35 - DNA, 19 bp, Artificial Sequence
  Description of Artificial Sequence Synthetic oligonucleotide
catgtggcaccgtttgcct

SEQ ID NO: 36 - DNA, 21 bp, Artificial Sequence
  Description of Artificial Sequence Synthetic oligonucleotide
aactaccagaaaggtatacct

SEQ ID NO: 37 - DNA, 21 bp, Artificial Sequence
  Description of Artificial Sequence Synthetic oligonucleotide
ucacaguguccuuuauguatt

SEQ ID NO: 38 - DNA, 21 bp, Artificial Sequence
  Description of Artificial Sequence Synthetic oligonucleotide
gcaugaaccggaggcccautt

SEQ ID NO: 39 - DNA, 19 bp, Artificial Sequence
  Description of Artificial Sequence Synthetic oligonucleotide
ccggacagttccatgtata

SEQ ID NO: 40 - DNA, 58 bp, Artificial Sequence
  Description of Artificial Sequence Synthetic oligonucleotide
ccggcccaggctgattggatcatagctcgagctatgatccaatcagcctgggtttttg

SEQ ID NO: 41 - DNA, 58 bp, Artificial Sequence
  Description of Artificial Sequence Synthetic oligonucleotide
ccggcctgaagcgaacatcagatttctcgagaaatctgatgttcgcttcaggtttttg

SEQ ID NO: 42 - DNA, 58 bp, Artificial Sequence
  Description of Artificial Sequence Synthetic oligonucleotide
ccggcagctctgtttagcactgatactcgagtatcagtgctaaacagagctgtttttg

SEQ ID NO: 43 - DNA, 57 bp, Artificial Sequence
  Description of Artificial Sequence Synthetic oligonucleotide
ccggcactctgagaaagaaacttatctcgagataagtttctttctcagagtgttttt

SEQ ID NO: 44 - DNA, 58 bp, Artificial Sequence
  Description of Artificial Sequence Synthetic oligonucleotide
ccggttcagtataggttcgtttaaactcgagtttaaacgaacctatactgaatttttg

SEQ ID NO: 45 - DNA, 57 bp, Artificial Sequence
  Description of Artificial Sequence Synthetic oligonucleotide
ccggcctgcgttgtattatctggaactcgagttccagataatacaacgcaggttttt

SEQ ID NO: 46 - DNA, 57 bp, Artificial Sequence; identical to SEQ ID NO: 45
  Description of Artificial Sequence Synthetic oligonucleotide

SEQ ID NO: 47 - DNA, 58 bp, Artificial Sequence
  Description of Artificial Sequence Synthetic oligonucleotide
ccgggccaactggtatcaccttcaactcgagttgaaggtgataccagttggctttttg

SEQ ID NO: 48 - DNA, 58 bp, Artificial Sequence
  Description of Artificial Sequence Synthetic oligonucleotide
ccggcctgtcctttgagaactctcactcgagtgagagttctcaaaggacaggtttttg

SEQ ID NO: 49 - DNA, 58 bp, Artificial Sequence
  Description of Artificial Sequence Synthetic oligonucleotide
ccggccctcttccacagaagcataactcgagttatgcttctgtggaagagggtttttg

SEQ ID NO: 50 - DNA, 58 bp, Artificial Sequence
  Description of Artificial Sequence Synthetic oligonucleotide
ccggacaatggtcgtgtccaccaaactcgagtttggtggacacgaccattgttttttg

SEQ ID NO: 51 - DNA, 59 bp, Artificial Sequence
  Description of Artificial Sequence Synthetic oligonucleotide
ccgggtaagacatcacaatcccattctcgagaatgggattgtgatgtcttacttttttg

SEQ ID NO: 52 - DNA, 58 bp, Artificial Sequence
  Description of Artificial Sequence Synthetic oligonucleotide
ccggcgcgcagaagggcgacaagaactcgagttcttgtcgcccttctgcgcgtttttg

SEQ ID NO: 53 - DNA, 59 bp, Artificial Sequence
  Description of Artificial Sequence Synthetic oligonucleotide
ccgggagtggcagaagctcaactatctcgagatagttgagcttctgccactcttttttg

SEQ ID NO: 54 - DNA, 59 bp, Artificial Sequence
  Description of Artificial Sequence Synthetic oligonucleotide
ccgggccaaatccttatcaaggaaactcgagtttccttgataaggatttggcttttttg

SEQ ID NO: 55 - DNA, 58 bp, Artificial Sequence
  Description of Artificial Sequence Synthetic oligonucleotide
ccggcctctctgcatcactgaacttctcgagaagttcagtgatgcagagaggtttttg

SEQ ID NO: 56 - DNA, 57 bp, Artificial Sequence
  Description of Artificial Sequence Synthetic oligonucleotide
ccgggctaccgacaccaaatctattctcgagaatagatttggtgtcggtagcttttt

SEQ ID NO: 57 - DNA, 57 bp, Artificial Sequence
  Description of Artificial Sequence Synthetic oligonucleotide
ccggatatccaccactttaaccttactcgagtaaggttaaagtggtggatatttttt

SEQ ID NO: 58 - DNA, 58 bp, Artificial Sequence
  Description of Artificial Sequence Synthetic oligonucleotide
ccggggtgaccatcttccaacatagctcgagctatgttggaagatggtcacctttttg

SEQ ID NO: 59 - DNA, 57 bp, Artificial Sequence
  Description of Artificial Sequence Synthetic oligonucleotide
ccggcacgaccaacagatactataactcgagttatagtatctgttggtcgtgttttt

SEQ ID NO: 60 - DNA, 57 bp, Artificial Sequence
  Description of Artificial Sequence Synthetic oligonucleotide
ccggcctcagcctctgccgcatgtactcgagtacatgcggcagaggctgaggttttt

SEQ ID NO: 61 - DNA, 59 bp, Artificial Sequence
  Description of Artificial Sequence Synthetic oligonucleotide
ccggcagcaatgtatggcacgacatctcgagatgtcgtgccatacattgctgttttttg

SEQ ID NO: 62 - DNA, 58 bp, Artificial Sequence
  Description of Artificial Sequence Synthetic oligonucleotide
ccgggcactgaaacaaggccaaattctcgagaatttggccttgtttcagtgctttttg

SEQ ID NO: 63 - DNA, 57 bp, Artificial Sequence
  Description of Artificial Sequence Synthetic oligonucleotide
ccggagatgaagattatgagcgagactcgagtctcgctcataatcttcatctttttt

SEQ ID NO: 64 - DNA, 57 bp, Artificial Sequence
  Description of Artificial Sequence Synthetic oligonucleotide
ccgggttctcatccaacgatgccatctcgagatggcatcgttggatgagaacttttt

SEQ ID NO: 65 - DNA, 58 bp, Artificial Sequence
  Description of Artificial Sequence Synthetic oligonucleotide
ccggtactttctatgagaagcgtatctcgagatacgcttctcatagaaagtatttttg

SEQ ID NO: 66 - DNA, 58 bp, Artificial Sequence
  Description of Artificial Sequence Synthetic oligonucleotide
ccggagccactgagaacaactttaactcgagttaaagttgttctcagtggcttttttg

SEQ ID NO: 67 - DNA, 58 bp, Artificial Sequence
  Description of Artificial Sequence Synthetic oligonucleotide
ccgggtgtttcacctgtctcggaaactcgagtttccgagacaggtgaaacactttttg

SEQ ID NO: 68 - DNA, 57 bp, Artificial Sequence
  Description of Artificial Sequence Synthetic oligonucleotide
ccgggagctgtggaaagtgttggatctcgagatccaacactttccacagctcttttt

SEQ ID NO: 69 - DNA, 58 bp, Artificial Sequence
  Description of Artificial Sequence Synthetic oligonucleotide
ccggcctccaggcatttggcaaatactcgagtatttgccaaatgcctggaggtttttg

SEQ ID NO: 70 - DNA, 58 bp, Artificial Sequence
  Description of Artificial Sequence Synthetic oligonucleotide
ccggcaagcacctagcatgacaatgctcgagcattgtcatgctaggtgcttgtttttg

SEQ ID NO: 71 - DNA, 58 bp, Artificial Sequence
  Description of Artificial Sequence Synthetic oligonucleotide
ccggcctcctttctatctgctcactctcgagagtgagcagatagaaaggaggtttttg

SEQ ID NO: 72 - DNA, 57 bp, Artificial Sequence
  Description of Artificial Sequence Synthetic oligonucleotide
ccggcgccgctttctgccctttgaactcgagttcaaagggcagaaagcggcgttttt

SEQ ID NO: 73 - DNA, 57 bp, Artificial Sequence
  Description of Artificial Sequence Synthetic oligonucleotide
ccgggcagcacgtgcgcctccactactcgagtagtggaggcgcacgtgctgcttttt

SEQ ID NO: 74 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 75 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 76 - protein, 17 aa, Artificial Sequence
  Description of Artificial Sequence Synthetic peptide
QIEYLAKQIVDNAIQQA

SEQ ID NO: 77 - protein, 21 aa, Artificial Sequence
  Description of Artificial Sequence Synthetic peptide
CGQIEYLAKQIVDNAIQQAGC

SEQ ID NO: 78 - protein, 50 aa, Artificial Sequence
  Description of Artificial Sequence Synthetic polypeptide
SLRECELYVQKHNIQALLKDSIVQLCTARPERPMAFLREYFERLEKEEAK

SEQ ID NO: 79 - protein, 55 aa, Artificial Sequence
  Description of Artificial Sequence Synthetic polypeptide
MSCGGSLRECELYVQKHNIQALLKDSIVQLCTARPERPMAFLREYFERLEKEEAK

SEQ ID NO: 80 - protein, 23 aa, Artificial Sequence
  Description of Artificial Sequence Synthetic peptide
CGFEELAWKIAKMIWSDVFQQGC

SEQ ID NO: 81 - protein, 51 aa, Artificial Sequence
  Description of Artificial Sequence Synthetic polypeptide
SLRECELYVQKHNIQALLKDVSIVQLCTARPERPMAFLREYFEKLEKEEAK

SEQ ID NO: 82 - protein, 54 aa, Artificial Sequence
  Description of Artificial Sequence Synthetic polypeptide
SLKGCELYVQLHGIQQVLKDCIVHLCISKPERPMKFLREHFEKLEKEENRQILA

SEQ ID NO: 83 - protein, 44 aa, Artificial Sequence
  Description of Artificial Sequence Synthetic polypeptide
SHIQIPPGLTELLQGYTVEVGQQPPDLVDFAVEYFTRLREARRQ

SEQ ID NO: 84 - protein, 44 aa, Artificial Sequence
  Description of Artificial Sequence Synthetic polypeptide
SIEIPAGLTELLQGFTVEVLRHQPADLLEFALQHFTRLQQENER

SEQ ID NO: 85 - DNA, 20 bp, Artificial Sequence
  Description of Artificial Sequence Synthetic primer
actggggtccttccatctct

SEQ ID NO: 86 - DNA, 19 bp, Artificial Sequence
  Description of Artificial Sequence Synthetic primer
cacaacaagagctcccatt

(19) United States
(12) Patent Application Publication
(10) Pub. No.: US 2016/0060707 A1
(43) Pub. Date: Mar. 3, 2016

(54) Identification of Cancer Genes by In-Vivo Fusion of Human Cancer Cells and Animal Cells

(71) Applicant: Immunomedics, Inc., Morris Plains, NJ (US)

(72) Inventors: David M. Goldenberg, Mendham, NJ (US); Chien-Hsing Chang, Downingtown, PA (US)

(21) Appl. No.: 14/836,064

(22) Filed: Aug. 26, 2015

Related U.S. Application Data
(60) Provisional application No. 62/043,601, filed on Aug. 29, 2014.

(51) Int. Cl. C12Q 1/68

(52) U.S. Cl. CPC C12Q 1/6886; C12Q 2600/158; C12Q 2600/106

(57) ABSTRACT

The present invention concerns compositions and methods for detecting and identifying novel cancer genes. The technique involves in vivo fusion of human cancer cells and animal cells, preferably hamster stromal cells, to form hybrid human cancer-animal cells, followed by identification of genes that are overexpressed in the hybrid cells compared to normal or transformed animal cells. The novel oncogenes or their protein products may be utilized for detection and/or diagnosis of human cancer or for development of new cancer therapies targeted against the novel oncogenes or their expressed proteins.

### RELATED APPLICATIONS

This application claims the benefit under 35 U.S.C. 119(e) of provisional U.S. Patent Application Ser. No. 62/043,601, filed Aug. 29, 2014, the entire text of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Aug. 21, 2015, is named IMM347US1_SL and is 29,709 bytes in size.

### FIELD OF THE INVENTION

The present invention concerns compositions and methods for detection and identification of genes (oncogenes) related to the induction, progression, metastasis and/or invasiveness of cancer in humans. Preferably, the methods involve fusion of primary human cancer cells with animal cells, such as hamster stromal cells, followed by propagation of the fused cells and analysis of gene expression profiles. Cancer genes may be detected by comparison of gene expression profiles in the hybrid cells, compared with animal cell controls. Identification of genes that are expressed in the hybrid cancer cells may be followed by mapping of the putative cancer genes to corresponding human chromosomes. Using heterospecific in-vivo cell fusion, genes encoding oncogenic and organogenic traits may be identified. The identified genes may provide novel targets for cancer therapy.

### BACKGROUND

Primary human tumor transplants, particularly to immunosuppressed rodents, such as nude and NOD/SCID mice, are used as preclinical models for evaluating tumor biology and drug sensitivity (Giovanella et al., 1978, Cancer 42:2269-81; Fidler et al., 1986, Cancer Metastasis Rev 5:29-49; DeRose et al., 2011, Nat Med 17:1514-20; Julien et al., 2012, Clin Cancer Res, 2012, 18:5314-28; Rubio-Viqueira & Hidalgo, 2008, Clin Pharmacol Ther 85:217-21; Sausville & Burger, 2006, Cancer Res 73:5315-19; Siolas & Hannon, 2013, Cancer Res 73:5315-19). These studies are based on the supposition that such xenografts retain the properties and critical genotypes of their donor tumors, thus being predictive for clinical translation. However, we and others have demonstrated that such transplants can induce tumors in their rodent recipients, such as golden hamsters (Goldenberg et al., 1967, Eur J Cancer 3:315-19; Lampert et al., 1968, Arch Geschwulstforsch 32:309-21; Fisher et al., 1970, Cancer 25:1286-1300), nude/SCID mice (Goldenberg & Pavia, 1981, Science 212:65-67; Goldenberg & Pavia, 1981, N Engl J Med 305:283-84; Goldenberg & Pavia, 1982, Proc Natl Acad Sci USA 79:2389-92; Popescu et al., 1970, Eur J Cancer 3:175-80; Bowen et al., 1983, In Vitro 19:635-41; Staab et al., 1983, J Cancer Res Clin Oncol 106:27-35; Russell et al., 1990, Int J Cancer 46:299-309; Ozen et al., 1997, Oncol Res 9:433-38; Pathak et al., 1997, Br J Cancer 76:1134-38), and immunosuppressed rats (Huebner et al., 1979, Proc Natl Acad Sci USA 76:1793-4), although infrequently (either because of low incidence or rare testing).

One plausible explanation is the horizontal transfer of oncogenic DNA (Huebner et al., 1979, Proc Natl Acad Sci USA 76:1793-4; Krontiris & Cooper, 1981, Proc Natl Acad Sci USA 78:1181-84; Garcia-Olmo & Garcia-Olmo, 2013, Crit Rev Oncogen 18:153-61). Indeed, lateral oncogenesis between tumor and its stromal cells can be traced back to Ehrlich and Apolant in 1905, who showed that stromal cells of a tumor can become a sarcoma when a carcinoma is grafted in mice, and in fact the authors conjectured that a chemical factor was implicated (Ehrlich et al., 1905, Berl Klin Wochenschr 42:871-74). Seventy-six years later, a human carcinoma transplanted to nude mice also was reported to induce fibrosarcomas that killed the nude mouse recipients and could propagate as malignant tumors in immune competent mice of the same genetic background (Goldenberg & Pavia, 1981, Science 212:65-67). In addition, a human ovarian cancer transplant to nude mice showed two cancer populations, an epithelial and a sarcomatous, the former showing human and the latter murine properties (Goldenberg & Pavia, 1982, Proc Natl Acad Sci USA 79:2389-92), suggesting lateral transduction or DNA transfer. Only the murine sarcoma cells, which were postulated to be induced by the human carcinoma cells, were metastatic and lethal in nude mice or immunocompetent mice of the same genetic background (Goldenberg & Pavia, 1982, Proc Natl Acad Sci USA 79:2389-92). This induction of stromal tumors in host animals after xenotransplantation of human epithelial cancers has been confirmed by others (Popescu et al., 1970, Eur J Cancer 3:175-80; Bowen et al., 1983, In Vitro 19:635-41; Staab et al., 1983, J Cancer Res Clin Oncol 106:27-35; Russell et al., 1990, Int J Cancer 46:299-309; Ozen et al., 1997, Oncol Res 9:433-38; Pathak et al., 1997, Br J Cancer 76:1134-38; Gupta et al., 1987, Cancer Res 47:5194-5201; Huebner et al., 1979, Proc Natl Acad Sci USA 76:1793-4), thus suggesting that cancer xenografts be carefully evaluated for horizontal oncogenesis (Goldenberg & Pavia, 1981, N Engl J Med 305:283-84; Pathak et al., 1998, Cancer 83:1891-93). How this transformation or induction occurred was not elucidated, but a viral role has been discussed (Bowen et al., 1983, In Vitro 19:635-41).

In some of these experiments involving primary human tumor transplants, transfer of functional human genetic information by in-vivo cell hybridization of the donor tumor and recipient host cells, showing chromosomal, immunological, or genetic features of both partners (Lampert et al., 1968, Arch Geschwulstforsch 32:309-21; Goldenberg & Gotz, 1968, Eur J Cancer 4:547-48; Gotz & Goldenberg, 1968, Experientia 24:957-58; Goldenberg, 1971, Exp Mol Pathol 14:134037; Goldenberg et al., 1971, Cancer Res 31:1148-52; Goldenberg et al., 1974, Nature 250:649-51), was proposed as the mechanism for induction of these tumors that exhibited highly invasive and metastatic behavior in their animal hosts (Goldenberg, 1983, Klin Wochenschr 46:898-99; Goldenberg, 2012, Expert Opin Bio Ther 12(Suppl 1):S133-39). For example, we reported that after long-term propagation of human-hamster hybrid tumors derived from a glioblastoma multiforme (Goldenberg et al., 1974, Nature 250:649-51) and two Hodgkin lymphomas, human DNA and genes could be confirmed by fluorescence in-situ hybridization (FISH) and polymerase chain reaction (PCR), and their donor organoid features by histology (Goldenberg et al., 2012, Int J Cancer 131:49-58; Goldenberg et al., 2013, PLoS One 8:e55324). Translation of some of these gene products was found by immunohistochemistry (IHC) in the glioblastoma multiforme transplants, even after propagation for over a year (Goldenberg et al., 2012, Int J Cancer 131:49-58).

A need exists for improved methods of detecting, identifying and/or mapping human oncogenes, by analyzing gene expression in hybrid cells produced by fusion of primary human cancer cells and animal stromal cells. Such novel oncogenes may be targeted for therapeutic intervention in cancer.

### SUMMARY

In various embodiments, the present invention concerns methods and compositions for detecting, identifying and/or mapping human cancer genes (oncogenes), involving in vivo fusion of human cancer cells with animal cells, preferably stromal cells. Preferably, the cancer cells are primary human cancer cells. In more preferred embodiments, the animal cells may be rodent cells, such as mouse, rat or hamster cells. The fusion creates a hybrid human cancer-animal cell that retains phenotypic characteristics of the parental cancer cell, such as malignancy, invasion and/or metastasis. The hybrid cell also continues to express human oncogenes.

In preferred embodiments, novel human oncogenes may be identified by analyzing gene expression profiles of the hybrid cells. More preferably, gene expression is compared between the hybrid cell and control animal cells. The animal cells may be normal cells or cancer cells. Methods of analyzing and comparing gene expression profiles are well known in the art and any such known methods may be used. For example, mRNA may be isolated from hybrid cells using commercial kits available from many manufacturers (e.g., Qiagen RNEASY® FFPE Kit, Qiagen, Germantown, Md.; Magnetic mRNA Isolation Kit, New England Biolabs, Ipswich, Mass.; GENELUTE™ mRNA Miniprep kit, Sigma-Aldrich, St. Louis, Mo.) and converted to cDNA using reverse transcriptase. Alternatively, kits are commercially available for comparison of gene expression without a separate mRNA isolation step (e.g., AMBION® CELLS-TO-C_{T}™ kit, Thermo Fisher Scientific, Grand Island, N.Y.). Quantification of cDNA species may be performed by standard techniques, such as oligonucleotide array hybridization (e.g., GENECHIP® Human U133_X3P Array, AFFYMETRIX®, Santa Clara, Calif.). Hybridized cDNA may be detected and quantified, for example, by fluorescence staining and use of a high-resolution laser scanner. Analysis and comparison of gene expression profiles may be performed using standard software (e.g. EXPRESSION CONSOLE™ Software, AFFYMETRIX®, Santa Clara, Calif.). Such methods, equipment and techniques are well known in the art and commercially available from many sources and any such known methodology may be used in the practice of the invention.

Preferably, gene expression profiles may be compared from multiple samples of hybrid cells and genes that are overexpressed in multiple independent hybrid cells may be identified. A cut-off value for the degree of overexpression in the hybrid cells, compared to animal control cells, may be selected (e.g., at least a two-fold increase in expression in the hybrid cell compared to control cell). The putative oncogenes may be mapped by comparison with database sequences of known human genes (e.g., NCBI Gene database, Bethesda, Md.). Gene function may also be identified by identification with known gene sequences or sequence homology comparison with genes of known function.

Identified oncogenes may be targeted for therapeutic intervention by known techniques, for example using interference RNA as discussed in more detail below. Alternatively, the protein products of putative oncogenes may be targeted using therapeutic antibodies. As discussed below, methods of making antibodies against any known protein or peptide sequence are routine in the art. Where the putative oncogenic protein is similar or identical to a known gene product, existing antibodies that are known to bind to that product may also be used. In still other alternative embodiments, known techniques such as combinatorial chemistry may be utilized to design novel inhibitors of the oncogenic protein.

Other embodiments concern cancer cell-targeting therapeutic immunoconjugates comprising an antibody or fragment thereof or fusion protein bound to at least one therapeutic agent. Preferably, the therapeutic agent is selected from the group consisting of a radionuclide, an immunomodulator, a hormone, a hormone antagonist, an enzyme, an oligonucleotide such as an anti-sense oligonucleotide or a siRNA, an enzyme inhibitor, a photoactive therapeutic agent, a cytotoxic agent such as a drug or toxin, an angiogenesis inhibitor and a pro-apoptotic agent. In embodiments where more than one therapeutic agent is used, the therapeutic agents may comprise multiple copies of the same therapeutic agent or else combinations of different therapeutic agents. The therapeutic antibody or immunoconjugate may be administered either alone or in combination with one or more other therapeutic agents.

In certain embodiments, the therapeutic agent is a cytotoxic agent, such as a drug or a toxin. Also preferred, the drug is selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicins and their analogs, antimetabolites, alkylating agents, antimitotics, anti-angiogenic agents, tyrosine kinase inhibitors, Bruton tyrosine kinase inhibitors, mTOR inhibitors, heat shock protein (HSP90) inhibitors, proteosome inhibitors, HDAC inhibitors, pro-apoptotic agents, methotrexate, CPT-11, SN-38, 2-PDOX, pro-2-PDOX, and a combination thereof.

In another preferred embodiment, the therapeutic agent is a toxin selected from the group consisting of ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, and Pseudomonas endotoxin and combinations thereof. Or an immunomodulator selected from the group consisting of a cytokine, a stem cell growth factor, a lymphotoxin, a hematopoietic factor, a colony stimulating factor (CSF), an interferon (IFN), a stem cell growth factor, erythropoietin, thrombopoietin and a combinations thereof.

Alternatively, the therapeutic agent is an enzyme selected from the group consisting of malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

Other therapeutic agents include radionuclides such as ¹⁴C, ¹³N, ¹⁵O, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁶⁷Ga, ⁷⁵Br, ⁷⁵Se, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo, ^{99m}Tc, ^{103m}Rh, ¹⁰³Rh, ¹⁰⁵Rh, ¹⁰⁵Rh, ¹⁰⁷Hg, ¹⁰⁹Pd, ¹⁰⁹Pt, ¹¹¹Ag, ¹¹¹In, ^{113m}In, ¹¹⁹Sb, ^{121m}Te, ^{122m}Te, ¹²⁵I, ^{125m}Te, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Rd, ²²⁴Ac, ²²⁵Ac, ²⁵⁵Fm or ²²⁷Th.

A variety of tyrosine kinase inhibitors are known in the art and any such known therapeutic agent may be utilized. Exemplary tyrosine kinase inhibitors include, but are not limited to canertinib, dasatinib, erlotinib, gefitinib, imatinib, lapatinib, leflunomide, nilotinib, pazopanib, semaxinib, sorafenib, sunitinib, sutent and vatalanib. A specific class of tyrosine kinase inhibitor is the Bruton tyrosine kinase inhibitor. Bruton tyrosine kinase (Btk) has a well-defined role in B-cell development. Bruton kinase inhibitors include, but are not limited to, PCI-32765 (ibrutinib), PCI-45292, GDC-0834, LFM-A13 and RN486.

An antibody or fragment may be conjugated to at least one diagnostic (or detection) agent. Preferably, the diagnostic agent is selected from the group consisting of a radionuclide, a contrast agent, a fluorescent agent, a chemiluminescent agent, a bioluminescent agent, a paramagnetic ion, an enzyme and a photoactive diagnostic agent. Still more preferred, the diagnostic agent is a radionuclide with an energy between 20 and 4,000 keV or is a radionuclide selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ⁸²mRb, ⁸³Sr, or other gamma-, beta-, or positron-emitters. In a particularly preferred embodiment, the diagnostic radionuclide ¹⁸F is used for labeling and PET imaging. The ¹⁸F may be attached to an antibody, antibody fragment or peptide by complexation to a metal, such as aluminum, and binding of the ¹⁸F-metal complex to a chelating moiety that is conjugated to a targeting protein, peptide or other molecule.

Also preferred, the diagnostic agent is a paramagnetic ion, such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), or a radiopaque material, such as barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

In still other embodiments, the diagnostic agent is a fluorescent labeling compound selected from the group consisting of fluorescein isothiocyanate, rhodamine, phycoerytherin, phycocyanin, allophycocyanin, o-phthaldehyde and fluorescamine, a chemiluminescent labeling compound selected from the group consisting of luminol, isoluminol, an aromatic acridinium ester, an imidazole, an acridinium salt and an oxalate ester, or a bioluminescent compound selected from the group consisting of luciferin, luciferase and aequorin. In another embodiment, a diagnostic immunoconjugate is used in intraoperative, endoscopic, or intravascular tumor diagnosis.

In certain embodiments, a novel putative oncogene identified by the techniques disclosed herein may be used to detect and/or diagnosis cancer, for example by detecting overexpression of the putative oncogene in a cell or tissue sample from an individual suspected of having cancer.

### BRIEF DESCRIPTION OF THE DRAWINGS

The following drawings are provided to illustrate preferred embodiments of the invention. However, the claimed subject matter is in no way limited by the illustrative embodiments disclosed in the drawings.

FIG. 1. Clustered heat map of the 39 human probe sets detected in all four hybrid tumor samples. The heat map depicts expression signal values for 39 AFFYMETRIX® Human U133_X3P probe sets detected in FFPE (formalin-fixed paraffin-embedded) sections from all four hybrids tested (IMM001-004) and a hamster control (IMM006). The corresponding samples were: IMM001=GW-532-Gen-2, IMM002=GW-532-Gen-34, IMM003=GW-584-Gen-28, IMM004=GB-749-Gen-4, IMM006=CCL-49. Prior to unsupervised hierarchical clustering, the MAS 5.0 signal values were log 2-transformed and row mean centered. Samples were clustered by Complete Linkage based on Pearson correlation; probe sets were clustered by Complete Linkage based on Euclidean distance. Criteria for detectable human gene expression included MAS 5.0 Detection p-values ≦0.065 in the hybrid sample and >0.065 in the hamster control, and ≧2-fold increased signal in the hybrid sample vs. the hamster control.

FIG. 2. PCR of human alpha satellite DNA. The presence of human DNA was demonstrated by the detection of the 171-bp product in GW-532 generation 52 (32 ng, lane 2), GW-532 generation 82 (52 ng, lane 3), GB-749 generation 2 (72 ng, lane 5), and GW-584 generation 3 (52 ng, lane 6), but not in the negative control of hamster melanoma, CCL-49 (60 ng, lane 8). Other lanes without a 171-bp product were GW-532 generation 11 (30 ng, lane 1), GB-749 generation 2 (42 ng, lane 4), and primers only (lane 9). Control human DNA from the Raji cell line (20 ng, lane 7) also shows the 171-bp product as expected. Lane M shows 100-bp ladder DNA MW markers. Primers used for amplification of human a satellite DNA were CATCACAAAGAAGTTTCTGAGAATGCTTC (SEQ ID NO:1, forward primer) and TGCATTCAACTCACAGAGTTGAACCTTCC (SEQ ID NO:2, reverse primer). The 171-bp and its higher oligomers were detected in the positive control of human Raji lymphoma cells (lane 7). The PCR conditions were denaturation at 94° C. for 5 min, followed 50 cycles of amplification at 94° C. for 30 sec, 60° C. for 30 sec, and 72° C. for 30 sec, followed by 72° C. for 10 min.

FIG. 3. One-step reverse transcription PCR. The mRNA transcripts of the FUR gene were detectable in GW-532 generation 11 (2031 ng, lane 1), GW-584 generation 3 (1230 ng, lane 2), and the positive control of human HepG2 cells (300 ng, lane 6), but not in the negative control hamster spleen cells (600 ng, lane 5). The 141-bp product was not observed in GW-532 generation 52 (1839 ng, lane 3), GW-532 generation 82 (1119 ng, lane 4) or with primer only (lane 7). Reverse transcription occurred at 55° C. for 20 min. The PCR conditions were denaturation at 94° C. for 2 min, followed 50 cycles of amplification at 94° C. for 15 sec, 56° C. for 30 sec, and 68° C. for 30 sec, followed by 68° C. for 5 min. Primers used for PCR amplification of F11R were CACAACAAGAGCTCCCATT (SEQ ID NO:3, forward primer) and ACTGGGGTCCTTCCATCTCT (SEQ ID NO:4, reverse primer).

FIG. 4. Additional one-step reverse transcription PCR. The mRNA transcripts of the F11R gene were detectable in GW-532 generation 11 (2031 ng, lane 1), GW-584 generation 3 (1230 ng, lane 2), and the positive control of HepG2 cells (300 ng, lane 5), whereas the target 141-bp was apparently absent in GW-532 generation 52 (1839 ng, lane 3), the negative control of hamster melanoma CCL-49 cells (2250 ng, lane 4), and with primers only (lane 6). Primers used and PCR amplification conditions were as disclosed in the legend to FIG. 3.

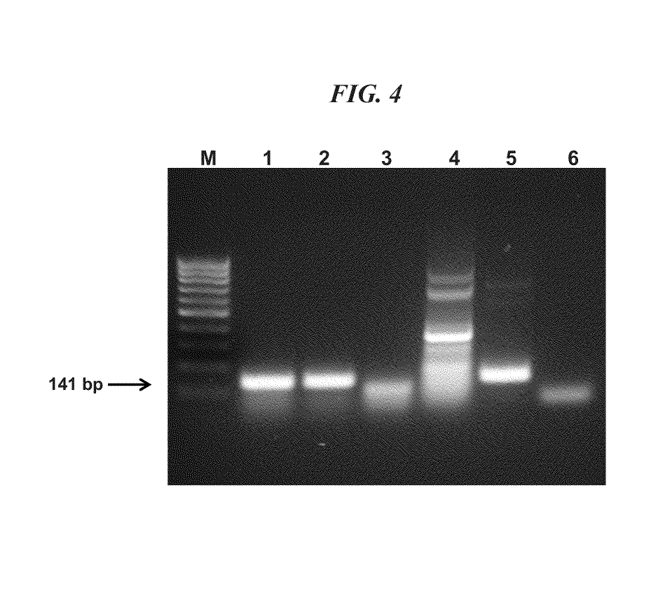

### DEFINITIONS

In the description that follows, a number of terms are used and the following definitions are provided to facilitate understanding of the claimed subject matter. Terms that are not expressly defined herein are used in accordance with their plain and ordinary meanings.

Unless otherwise specified, a or an means “one or more.”

The term about is used herein to mean plus or minus ten percent (10%) of a value. For example, “about 100” refers to any number between 90 and 110.

An antibody, as used herein, refers to a full-length (i.e., naturally occurring or formed by normal immunoglobulin gene fragment recombinatorial processes) immunoglobulin molecule (e.g., an IgG antibody) or an antigen-binding portion of an immunoglobulin molecule, such as an antibody fragment. An antibody or antibody fragment may be conjugated or otherwise derivatized within the scope of the claimed subject matter. Such antibodies include but are not limited to IgG1, IgG2, IgG3, IgG4 (and IgG4 subforms), as well as IgA isotypes. In preferred embodiments, antibodies and antibody fragments are selected to bind to human antigens.

An antibody fragment is a portion of an antibody such as F(ab′)₂, F(ab)₂, Fab′, Fab, Fv, scFv (single chain Fv), single domain antibodies (DABs or VHHs) and the like, including the half-molecules of IgG4 cited above (van der Neut Kolfschoten et al. (Science 2007; 317(14 September):1554-1557). A commercially available form of single domain antibody is referred to as a nanobody (ABLYNX®, Ghent, Belgium). Regardless of structure, an antibody fragment of use binds with the same antigen that is recognized by the intact antibody. The term “antibody fragment” also includes synthetic or genetically engineered proteins that act like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”). The Fv fragments may be constructed in different ways to yield multivalent and/or multispecific binding forms. In the case of multivalent, they have more than one binding site against the specific epitope, whereas with multispecific forms, more than one epitope (either of the same antigen or against one antigen and a different antigen) is bound.

A naked antibody is generally an entire antibody that is not conjugated to a therapeutic agent. This is so because the Fc portion of the antibody molecule provides effector or immunological functions, such as complement fixation and ADCC (antibody-dependent cell cytotoxicity), which set mechanisms into action that may result in cell lysis. However, the Fc portion may not be required for therapeutic function of the antibody, but rather other mechanisms, such as apoptosis, anti-angiogenesis, anti-metastatic activity, anti-adhesion activity, such as inhibition of heterotypic or homotypic adhesion, and interference in signaling pathways, may come into play and interfere with disease progression. Naked antibodies include both polyclonal and monoclonal antibodies, and fragments thereof, that include murine antibodies, as well as certain recombinant antibodies, such as chimeric, humanized or human antibodies and fragments thereof. As used herein, “naked” is synonymous with “unconjugated,” and means not linked or conjugated to a therapeutic agent.

A chimeric antibody is a recombinant protein that contains the variable domains of both the heavy and light antibody chains, including the complementarity determining regions (CDRs) of an antibody derived from one species, preferably a rodent antibody, more preferably a murine antibody, while the constant domains of the antibody molecule are derived from those of a human antibody. For veterinary applications, the constant domains of the chimeric antibody may be derived from that of other species, such as a primate, cat or dog.

A humanized antibody is a recombinant protein in which the CDRs from an antibody from one species; e.g., a murine antibody, are transferred from the heavy and light variable chains of the murine antibody into human heavy and light variable domains (framework regions). The constant domains of the antibody molecule are derived from those of a human antibody. In some cases, specific residues of the framework region of the humanized antibody, particularly those that are touching or close to the CDR sequences, may be modified, for example replaced with the corresponding residues from the original murine, rodent, subhuman primate, or other antibody.

A human antibody is an antibody obtained, for example, from transgenic mice that have been “engineered” to produce human antibodies in response to antigenic challenge. In this technique, elements of the human heavy and light chain loci are introduced into strains of mice derived from embryonic stem cell lines that contain targeted disruptions of the endogenous heavy chain and light chain loci. The transgenic mice can synthesize human antibodies specific for various antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, the Examples section of each of which is incorporated herein by reference.

A therapeutic agent is a molecule or atom that is useful in the treatment of a disease. Examples of therapeutic agents include, but are not limited to, antibodies, antibody fragments, conjugates, drugs, cytotoxic agents, proapoptotic agents, toxins, nucleases (including DNAses and RNAses), hormones, immunomodulators, chelators, boron compounds, photoactive agents or dyes, radioisotopes or radionuclides, oligonucleotides, interference RNA, peptides, anti-angiogenic agents, chemotherapeutic agents, cyokines, chemokines, prodrugs, enzymes, binding proteins or peptides or combinations thereof.

An immunoconjugate is an antibody, antibody fragment or other antibody moiety conjugated to a therapeutic agent.

As used herein, the term antibody fusion protein is a recombinantly-produced antigen-binding molecule in which one or more natural antibodies, single-chain antibodies or antibody fragments are linked to another moiety, such as a protein or peptide, a toxin, a cytokine, a hormone, etc. In certain preferred embodiments, the fusion protein may comprise two or more of the same or different antibodies, antibody fragments or single-chain antibodies fused together, which may bind to the same epitope, different epitopes on the same antigen, or different antigens.

An immunomodulator is a therapeutic agent that when present, alters, suppresses or stimulates the body's immune system. Typically, an immunomodulator of use stimulates immune cells to proliferate or become activated in an immune response cascade, such as macrophages, dendritic cells, B-cells, and/or T-cells. An example of an immunomodulator as described herein is a cytokine, which is a soluble small protein of approximately 5-20 kDa that is released by one cell population (e.g., primed T-lymphocytes) on contact with specific antigens, and which acts as an intercellular mediator between cells. As the skilled artisan will understand, examples of cytokines include lymphokines, monokines, interleukins, and several related signaling molecules, such as tumor necrosis factor (TNF) and interferons. Chemokines are a subset of cytokines Certain interleukins and interferons are examples of cytokines that stimulate T cell or other immune cell proliferation.

### Gene Expression

Certain embodiments concern techniques for analyzing and comparing levels of gene expression to identify human genes that are overexpressed in hybrid human cancer-animal cells, such as human cancer-hamster stromal hybrid cells. Gene expression in the hybrid cell is compared to gene expression in control cells, for example in normal animal cells or animal cancer cells. Genes that are overexpressed in the hybrid cells compared to control cells are identified as putative cancer genes (oncogenes).

Various techniques are known in the art for analyzing gene expression and any such known technique may be utilized. One of the earlier techniques involved Northern blotting (see, e.g., Kevil et al. 1997, Biochem Biophys Res Commun 238:277-79), in which a sample of RNA is size-fractionated by agarose gel electrophoresis. Following transfer to a membrane, the RNA is hybridized with a labeled probe and an image is developed by autoradiography, colorimetric or chemiluminescent staining. While suitable for measuring expression levels of selected known genes, the technique is cumbersome for large-scale gene expression screening as practiced in the instant methods.

An alternative technique is quantitative RT-PCR (see, e.g., Radonic et al., 2004, Biochem Biophys Res Commun 313:856-62), involving reverse transcription followed by quantitative PCR. The method may start with separation of mRNA from other nucleic acids, for example by affinity column chromatography (e.g., oligo-dT column) or magnetic bead-based separation (e.g., oligo-dT magnetic beads). However, in alternative techniques mRNA separation is not performed and the assay may utilize total RNA samples. PCR amplification requires the use of primers that can specifically hybridize to each gene product of interest. Another variation involves hybridization with DNA microarrays (biochips) (see, e.g., Maskos & Southern, 1992, Nucl Acids Res 10:1679-84). Each chip contains samples of nucleic acids attached to specific locations on the chip. Microarrays may contain probes against tens of thousands of genes per chip. Gene products that hybridize to the chip may be detected and quantified using amplified target DNA that has been labeled with a fluorescent, chemiluminescent or other detection agent. The fluorescent or luminescent signal can be quantified by measuring the light emission from each spot on the chip.

Other alternative techniques for gene expression analysis are known and may be utilized, such as serial analysis of gene expression (SAGE) (see, e.g., Velculescu et al., 1995, Science 270:484-87) or RNA-Seq (also known as whole transcriptome shotgun sequencing or WTSS) (see, e.g., Morin et al., 2008, Biotechniques 45:81-94). These and other alternative techniques may be performed using kits, equipment and/or software packages that may be obtained from numerous commercial sources. These include, but are not limited to, AMBION® CELLS-TO-C_{T}™ kit (Thermo Fisher Scientific, Grand Island, N.Y.); TAQMAN® Gene Expression CELLS-TO-C_{T}™ kit (Thermo Fisher Scientific, Grand Island, N.Y.); RT² REAL-TIME™ Gene Expression Assay Kit (Qiagen, Valencia, Calif.); AMBION® WT Expression Kit (AFFYMETRIX®, Santa Clara, Calif.); SIMPLE SCREEN™ Mammalian Gene Expression Kit (KempBio, Frederick, Md.); QUANTIGENE® 2.0 Assay (AFFYMETRIX®, Santa Clara, Calif.); GENECHIP® Human U133 X3P Array (AFFYMETRIX®, Santa Clara, Calif.); GENECHIP® PRIMEVIEW™ Human Gene Expression Array(AFFYMETRIX®, Santa Clara, Calif.); TISSUESCAN™ cDNA Arrays (OriGene, Rockville, Md.); NEXUS EXPRESSION™ SOFTWARE (BioDiscovery, Hawthorne, Calif.); AFFYMETRIX® EXPRESSION CONSOLE™ Software (AFFYMETRIX®, Santa Clara, Calif.); ExpressionSuite Software (Thermo Fisher Scientific, Rockford, Ill.); and many others. These and other commercially available kits, apparatus, and software may be used for analysis and comparison of gene expression profiles within the scope of the claimed methods and compositions.

### Inhibitory RNA

In various embodiments, inhibitory RNA species, such as RNAi or siRNA, that are directed against oncogenes identified by the claimed methods may be used to treat cancer. A preferred form of therapeutic oligonucleotide is siRNA. The skilled artisan will realize that any siRNA or interference RNA species may be delivered to a cancer cell tissue. Many siRNA species against a wide variety of targets are known in the art, and any such known siRNA may be utilized. Techniques for developing and using inhibitory oligonucleotides against newly discovered oncogenes are also known in the art (see, e.g., Elbashir et al., 2001, Nature 411:494-8; Tabernero et al., 2013, Cancer Discovery 3:406-17; Geisbert et al., 2010, Lancet 375:1896-1905).

A wide variety of siRNA species are available from commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Mirus Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL complexes.

Exemplary siRNA species known in the art are listed in Table 1. Although siRNA is delivered as a double-stranded molecule, for simplicity only the sense strand sequences are shown in Table 1.

TABLE 1
Exemplary siRNA Sequences

| | | SEQ ID |
|---|---|---|
| Target | Sequence | NO |
| VEGF R2 | AATGCGGCGGTGGTGACAGTA | SEQ ID |
| | | NO: 8 |
| VEGF R2 | AAGCTCAGCACACAGAAAGAC | SEQ ID |
| | | NO: 9 |
| CXCR4 | UAAAAUCUUCCUGCCCACCdTdT | SEQ ID |
| | | NO: 10 |
| CXCR4 | GGAAGCUGUUGGCUGAAAAdTdT | SEQ ID |
| | | NO: 11 |
| PPARC1 | AAGACCAGCCUCUUUGCCCAG | SEQ ID |
| | | NO: 12 |
| Dynamin 2 | GGACCAGGCAGAAAACGAG | SEQ ID |
| | | NO: 13 |
| Catenin | CUAUCAGGAUGACGCGG | SEQ ID |
| | | NO: 14 |
| E1A binding | UGACACAGGCAGGCUUGACUU | SEQ ID |
| protein | | NO: 15 |
| Plasminogen | GGTGAAGAAGGGCGTCCAA | SEQ ID |
| activator | | NO: 16 |
| K-ras | GATCCGTTGGAGCTGTTGGCGTAGTT | SEQ ID |
| | CAAGAGACTCGCCAACAGCTCCAACT TT | NO: 17 |
| | TGGAAA | |
| Sortilin 1 | AGGTGGTGTTAACAGCAGAG | SEQ ID |
| | | NO: 18 |
| Apolipo- | AAGGTGGAGCAAGCGGTGGAG | SEQ ID |
| protein E | | NO: 19 |
| Apolipo- | AAGGAGTTGAAGGCCGACAAA | SEQ ID |
| protein E | | NO: 20 |
| Bcl-X | UAUGGAGCUGCAGAGGAUGdTdT | SEQ ID |
| | | NO: 21 |
| Raf-1 | TTTGAATATCTGTGCTGAGAACACA | SEQ ID |
| | GTTCTCAGCACAGATATTCTTTTT | NO: 22 |
| Heat shock | AATGAGAAAAGCAAAAGGTGCCCTGTCTC | SEQ ID |
| transcription | | NO: 23 |
| factor 2 | | |
| IGFBP3 | AAUCAUCAUCAAGAAAGGGCA | SEQ ID |
| | | NO: 24 |
| Thioredoxin | AUGACUGUCAGGAUGUUGCdTdT | SEQ ID |
| | | NO: 25 |
| CD44 | GAACGAAUCCUGAAGACAUCU | SEQ ID |
| | | NO: 26 |
| MMP14 | AAGCCTGGCTACAGCAATATGCCTGTCTC | SEQ ID |
| | | NO: 27 |
| MAPKAPK | UGACCAUCACCGAGUUUAUdTdT | SEQ ID |
| 2 | | NO: 28 |
| FGFR1 | AAGTCGGACGCAACAGAGAAA | SEQ ID |
| | | NO: 29 |
| ERBB2 | CUACCUUUCUACGGACGUGdTdT | SEQ ID |
| | | NO: 30 |
| BCL2L1 | CTGCCTAAGGCGGATTTGAAT | SEQ ID |
| | | NO: 31 |
| ABL1 | TTAUUCCUUCUUCGGGAAGUC | SEQ ID |
| | | NO: 32 |
| CEACAM1 | AACCTTCTGGAACCCGCCCAC | SEQ ID |
| | | NO: 33 |
| CD9 | GAGCATCTTCGAGCAAGAA | SEQ ID |
| | | NO: 34 |
| CD151 | CATGTGGCACCGTTTGCCT | SEQ ID |
| | | NO: 35 |
| Caspase 8 | AACTACCAGAAAGGTATACCT | SEQ ID |
| | | NO: 36 |
| BRCA1 | UCACAGUGUCCUUUAUGUAdTdT | SEQ ID |
| | | NO: 37 |
| p53 | GCAUGAACCGGAGGCCCAUTT | SEQ ID |
| | | NO: 38 |
| CEACAM6 | CCGGACAGTTCCATGTATA | SEQ ID |
| | | NO: 39 |
| ABCC6 | CCGGCCCAGGCTGATTGGATCATAGCTCG | SEQ ID |
| | AGCTATGATCCAATCAGCCTGGGTTTTTG | NO: 40 |
| CARD11 | CCGGCCTGAAGCGAACATCAGATTTCTCG | SEQ ID |
| | AGAAATCTGATGTTCGCTTCAGGTTTTTG | NO: 41 |
| CDH3 | CCGGCAGCTCTGTTTAGCACTGATACTCG | SEQ ID |
| | AGTATCAGTGCTAAACAGAGCTGTTTTTG | NO: 42 |
| CFLAR | CCGGCACTCTGAGAAAGAAACTTATCTCG | SEQ ID |
| | AGATAAGTTTCTTTCTCAGAGTGTTTTT | NO: 43 |
| DARS | CCGGTTCAGTATAGGTTCGTTTAAACTCG | SEQ ID |
| | AGTTTAAACGAACCTATACTGAATTTTTG | NO: 44 |
| DYSF | CCGGCCTGCGTTGTATTATCTGGAACTCG | SEQ ID |
| | AGTTCCAGATAATACAACGCAGGTTTTT | NO: 45 |
| ECEL1 | CCGGCCTGCGTTGTATTATCTGGAACTCG | SEQ ID |
| | AGTTCCAGATAATACAACGCAGGTTTTT | NO: 46 |
| F11R | CCGGGCCAACTGGTATCACCTTCAACTCG | SEQ ID |
| | AGTTGAAGGTGATACCAGTTGGCTTTTTG | NO: 47 |
| FUT7 | CCGGCCTGTCCTTTGAGAACTCTCACTCG | SEQ ID |
| | AGTGAGAGTTCTCAAAGGACAGGTTTTTG | NO: 48 |
| GPAT2 | CCGGCCCTCTTCCACAGAAGCATAACTCG | SEQ ID |
| | AGTTATGCTTCTGTGGAAGAGGGTTTTTG | NO: 49 |
| GTPBP6 | CCGGACAATGGTCGTGTCCACCAAACTCG | SEQ ID |
| | AGTTTGGTGGACACGACCATTGTTTTTTG | NO: 50 |
| GUSBP2 | CCGGGTAAGACATCACAATCCCATTCTCG | SEQ ID |
| | AGAATGGGATTGTGATGTCTTACTTTTTTG | NO: 51 |
| HOXB8 | CCGGCGCGCAGAAGGGCGACAAGAACTCG | SEQ ID |
| | AGTTCTTGTCGCCCTTCTGCGCGTTTTTG | NO: 52 |
| MREG | CCGGGAGTGGCAGAAGCTCAACTATCTCG | SEQ ID |
| | AGATAGTTGAGCTTCTGCCACTCTTTTTTG | NO: 53 |
| NAA40 | CCGGGCCAAATCCTTATCAAGGAAACTCG | SEQ ID |
| | AGTTTCCTTGATAAGGATTTGGCTTTTTTG | NO: 54 |
| PARP15 | CCGGCCTCTCTGCATCACTGAACTTCTCG | SEQ ID |
| | AGAAGTTCAGTGATGCAGAGAGGTTTTTG | NO: 55 |
| POU2F2 | CCGGGCTACCGACACCAAATCTATTCTCG | SEQ ID |
| | AGAATAGATTTGGTGTCGGTAGCTTTTT | NO: 56 |
| PPARA | CCGGATATCCACCACTTTAACCTTACTCG | SEQ ID |
| | AGTAAGGTTAAAGTGGTGGATATTTTTT | NO: 57 |
| PPP1R18 | CCGGGGTGACCATCTTCCAACATAGCTCG | SEQ ID |
| | AGCTATGTTGGAAGATGGTCACCTTTTTG | NO: 58 |
| PRKD2 | CCGGCACGACCAACAGATACTATAACTCG | SEQ ID |
| | AGTTATAGTATCTGTTGGTCGTGTTTTT | NO: 59 |
| PTGIR | CCGGCCTCAGCCTCTGCCGCATGTACTCG | SEQ ID |
| | AGTACATGCGGCAGAGGCTGAGGTTTTT | NO: 60 |
| PXMP4 | CCGGCAGCAATGTATGGCACGACATCTCG | SEQ ID |
| | AGATGTCGTGCCATACATTGCTGTTTTTTG | NO: 61 |
| QRSL1 | CCGGGCACTGAAACAAGGCCAAATTCTCG | SEQ ID |
| | AGAATTTGGCCTTGTTTCAGTGCTTTTTG | NO: 62 |
| RBM17 | CCGGAGATGAAGATTATGAGCGAGACTCG | SEQ ID |
| | AGTCTCGCTCATAATCTTCATCTTTTTT | NO: 63 |
| RGS9 | CCGGGTTCTCATCCAACGATGCCATCTCG | SEQ ID |
| | AGATGGCATCGTTGGATGAGAACTTTTT | NO: 64 |
| RPS6 | CCGGTACTTTCTATGAGAAGCGTATCTCG | SEQ ID |
| | AGATACGCTTCTCATAGAAAGTATTTTTG | NO: 65 |
| SEMA3F | CCGGAGCCACTGAGAACAACTTTAACTCG | SEQ ID |
| | AGTTAAAGTTGTTCTCAGTGGCTTTTTTG | NO: 66 |
| SLC9A5 | CCGGGTGTTTCACCTGTCTCGGAAACTCG | SEQ ID |
| | AGTTTCCGAGACAGGTGAAACACTTTTTG | NO: 67 |
| SSH3 | CCGGGAGCTGTGGAAAGTGTTGGATCTCG | SEQ ID |
| | AGATCCAACACTTTCCACAGCTCTTTTT | NO: 68 |
| TMEM184A | CCGGCCTCCAGGCATTTGGCAAATACTCG | SEQ ID |
| | AGTATTTGCCAAATGCCTGGAGGTTTTTG | NO: 69 |
| TSSK2 | CCGGCAAGCACCTAGCATGACAATGCTCG | SEQ ID |
| | AGCATTGTCATGCTAGGTGCTTGTTTTTG | NO: 70 |
| UBE2E1 | CCGGCCTCCTTTCTATCTGCTCACTCTCG | SEQ ID |
| | AGAGTGAGCAGATAGAAAGGAGGTTTTTG | NO: 71 |
| ZFHX2 | CCGGCGCCGCTTTCTGCCCTTTGAACTCG | SEQ ID |
| | AGTTCAAAGGGCAGAAAGCGGCGTTTTT | NO: 72 |
| ZNF580 | CCGGGCAGCACGTGCGCCTCCACTACTCG | SEQ ID |
| | AGTAGTGGAGGCGCACGTGCTGCTTTTT | NO: 73 |

The skilled artisan will realize that Table 1 represents a very small sampling of the total number of siRNA species known in the art, and that any such known siRNA may be utilized in the claimed methods and compositions.

### Antibody Techniques

Certain embodiments may involve novel cancer therapies, using antibodies or antigen-binding antibody fragments against the protein products of cancer genes discovered using the claimed methods and compositions. The antibodies or fragments thereof may induce cell death of cancer cells directly, for example by inducing an immune response against the targeted cell, or by delivering one or more therapeutic agents to the target cell as described in detail below. In alternative embodiments, antibodies or fragments against a known tumor-associated antigen may be used to deliver siRNA or RNAi species directed against newly identified oncogenes to a cancer cell.

Techniques for preparing monoclonal antibodies against virtually any target antigen are well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice with a composition comprising an antigen (preferably a human antigen), removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Various techniques, such as production of chimeric or humanized antibodies, may involve procedures of antibody cloning and construction. The antigen-binding Vκ (variable light chain) and V_{H} (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned V_{L} and V_{H} genes can be expressed in cell culture as a chimeric Ab as described by Orlandi et al., (Proc. Natl. Acad. Sci., USA, 86: 3833 (1989)). Based on the V gene sequences, a humanized antibody can then be designed and constructed as described by Leung et al. (Mol. Immunol., 32: 1413 (1995)).

cDNA can be prepared from any known hybridoma line or transfected cell line producing a murine antibody by general molecular cloning techniques (Sambrook et al., Molecular Cloning, A laboratory manual, 2^{nd} Ed (1989)). The Vκ sequence for the antibody may be amplified using the primers VK1BACK and VK1FOR (Orlandi et al., 1989) or the extended primer set described by Leung et al. (BioTechniques, 15: 286 (1993)). The V_{H} sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for V_{H} can be subcloned into a similar staging vector, such as the pBluescript-based VHpBS. Expression cassettes containing the Vκ and V_{H} sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and V_{H} expression cassettes can be excised and subcloned into a single expression vector, such as pdHL2, as described by Gillies et al. (J. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

### Chimeric and Humanized Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. Methods for constructing chimeric antibodies are well known in the art (e.g., Leung et al., 1994, Hybridoma 13:469).

A chimeric monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. To preserve the stability and antigen specificity of the humanized monoclonal, one or more human FR residues may be replaced by the mouse counterpart residues. Humanized monoclonal antibodies may be used for therapeutic treatment of subjects. Techniques for production of humanized monoclonal antibodies are well known in the art. (See, e.g., Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285; Sandhu, Crit. Rev. Biotech., 1992, 12:437; Tempest et al., 1991, Biotechnology 9:266; Singer et al., J. Immun., 1993, 150:2844.)

Other embodiments may concern non-human primate antibodies. General techniques for raising therapeutically useful antibodies in baboons may be found, for example, in Goldenberg et al., WO 91/11465 (1991), and in Losman et al., Int. J. Cancer 46: 310 (1990). In another embodiment, an antibody may be a human monoclonal antibody. Such antibodies may be obtained from transgenic mice that have been engineered to produce specific human antibodies in response to antigenic challenge, as discussed below.

### Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50; each incorporated herein by reference). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97, incorporated herein by reference). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

The XenoMouse® was transformed with germline-configured YACs (yeast artificial chromosomes) that contained portions of the human IgH and Ig kappa loci, including the majority of the variable region sequences, along accessory genes and regulatory sequences. The human variable region repertoire may be used to generate antibody producing B cells, which may be processed into hybridomas by known techniques. A XenoMouse® immunized with a target antigen will produce human antibodies by the normal immune response, which may be harvested and/or produced by standard techniques discussed above. A variety of strains of XenoMouse® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XenoMouse® system but may utilize any transgenic animal that has been genetically engineered to produce human antibodies.

### Production of Antibody Fragments

Antibody fragments may be obtained, for example, by pepsin or papain digestion of whole antibodies by conventional methods. For example, antibody fragments may be produced by enzymatic cleavage of antibodies with pepsin to provide a 5S fragment denoted F(ab′)₂. This fragment may be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment. Exemplary methods for producing antibody fragments are disclosed in U.S. Pat. No. 4,036,945; U.S. Pat. No. 4,331,647; Nisonoff et al., 1960, Arch Biochem Biophys, 89:230; Porter, 1959, Biochem. J., 73:119; Edelman et al., 1967, METHODS IN ENZYMOLOGY, page 422 (Academic Press), and Coligan et al. (eds.), 1991, CURRENT PROTOCOLS IN IMMUNOLOGY, (John Wiley & Sons).

Other methods of cleaving antibodies, such as separation of heavy chains to form monovalent light-heavy chain fragments, further cleavage of fragments or other enzymatic, chemical or genetic techniques also may be used, so long as the fragments bind to the antigen that is recognized by the intact antibody. For example, Fv fragments comprise an association of V_{H} and V_{L} chains. This association can be noncovalent, as described in Inbar et al., 1972, Proc. Nat'l. Acad. Sci. USA, 69:2659. Alternatively, the variable chains may be linked by an intermolecular disulfide bond or cross-linked by chemicals such as glutaraldehyde. See Sandhu, 1992, Crit. Rev. Biotech., 12:437.

Preferably, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains, connected by an oligonucleotides linker sequence. The structural gene is inserted into an expression vector that is subsequently introduced into a host cell, such as E. coli. The recombinant host cells synthesize a single polypeptide chain with a linker peptide bridging the two V domains. Methods for producing scFvs are well-known in the art. See Whitlow et al., 1991, Methods: A Companion to Methods in Enzymology 2:97; Bird et al., 1988, Science, 242:423; U.S. Pat. No. 4,946,778; Pack et al., 1993, Bio/Technology, 11:1271, and Sandhu, 1992, Crit. Rev. Biotech., 12:437.

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 06, 43:1912-19; Tanha et al., J. Biol. Chem. 2001, 276:24774-780). Other types of antibody fragments may comprise one or more complementarity-determining regions (CDRs). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See Larrick et al., 1991, Methods: A Companion to Methods in Enzymology 2:106; Ritter et al. (eds.), 1995, MONOCLONAL ANTIBODIES: PRODUCTION, ENGINEERING AND CLINICAL APPLICATION, pages 166-179 (Cambridge University Press); Birch et al., (eds.), 1995, MONOCLONAL ANTIBODIES: PRINCIPLES AND APPLICATIONS, pages 137-185 (Wiley-Liss, Inc.)

### Antibody Variations

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797; each incorporated herein by reference).

### Antibody Allotypes

Immunogenicity of therapeutic antibodies is associated with increased risk of infusion reactions and decreased duration of therapeutic response (Baert et al., 2003, N Engl J Med 348:602-08). The extent to which therapeutic antibodies induce an immune response in the host may be determined in part by the allotype of the antibody (Stickler et al., 2011, Genes and Immunity 12:213-21). Antibody allotype is related to amino acid sequence variations at specific locations in the constant region sequences of the antibody. The allotypes of IgG antibodies containing a heavy chain γ-type constant region are designated as Gm allotypes (1976, J Immunol 117:1056-59).

For the common IgG1 human antibodies, the most prevalent allotype is G1m1 (Stickler et al., 2011, Genes and Immunity 12:213-21). However, the G1m3 allotype also occurs frequently in Caucasians (Stickler et al., 2011). It has been reported that G1m1 antibodies contain allotypic sequences that tend to induce an immune response when administered to non-G1m1 (nG1m1) recipients, such as G1m3 patients (Stickler et al., 2011). Non-G1m1 allotype antibodies are not as immunogenic when administered to G1m1 patients (Stickler et al., 2011).

The human G1m1 allotype comprises the amino acids aspartic acid at Kabat position 356 and leucine at Kabat position 358 in the CH3 sequence of the heavy chain IgG1. The nG1m1 allotype comprises the amino acids glutamic acid at Kabat position 356 and methionine at Kabat position 358. Both G1m1 and nG1m1 allotypes comprise a glutamic acid residue at Kabat position 357 and the allotypes are sometimes referred to as DEL and EEM allotypes. A non-limiting example of the heavy chain constant region sequences for G1m1 and nG1m1 allotype antibodies is shown below for the exemplary antibodies rituximab (SEQ ID NO:5) and veltuzumab (SEQ ID NO:6).

Rituximab heavy chain variable region sequence
(SEQ ID NO: 5)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKAEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Veltuzumab heavy chain variable region
(SEQ ID NO: 6)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Jefferis and Lefranc (2009, mAbs 1:1-7) reviewed sequence variations characteristic of IgG allotypes and their effect on immunogenicity. They reported that the G1m3 allotype is characterized by an arginine residue at Kabat position 214, compared to a lysine residue at Kabat 214 in the G1m17 allotype. The nG1m1,2 allotype was characterized by glutamic acid at Kabat position 356, methionine at Kabat position 358 and alanine at Kabat position 431. The G1m1,2 allotype was characterized by aspartic acid at Kabat position 356, leucine at Kabat position 358 and glycine at Kabat position 431. In addition to heavy chain constant region sequence variants, Jefferis and Lefranc (2009) reported allotypic variants in the kappa light chain constant region, with the Km1 allotype characterized by valine at Kabat position 153 and leucine at Kabat position 191, the Km1,2 allotype by alanine at Kabat position 153 and leucine at Kabat position 191, and the Km3 allotypoe characterized by alanine at Kabat position 153 and valine at Kabat position 191.

With regard to therapeutic antibodies, veltuzumab and rituximab are, respectively, humanized and chimeric IgG1 antibodies against CD20, of use for therapy of a wide variety of hematological malignancies. Table 2 compares the allotype sequences of rituximab vs. veltuzumab. As shown in Table 2, rituximab (G1m17,1) is a DEL allotype IgG1, with an additional sequence variation at Kabat position 214 (heavy chain CH1) of lysine in rituximab vs. arginine in veltuzumab. It has been reported that veltuzumab is less immunogenic in subjects than rituximab (see, e.g., Morchhauser et al., 2009, J Clin Oncol 27:3346-53; Goldenberg et al., 2009, Blood 113:1062-70; Robak & Robak, 2011, BioDrugs 25:13-25), an effect that has been attributed to the difference between humanized and chimeric antibodies. However, the difference in allotypes between the EEM and DEL allotypes likely also accounts for the lower immunogenicity of veltuzumab.

TABLE 2
Allotypes of Rituximab vs. Veltuzumab

| | | Heavy chain position and associated allotypes | | | | | |
|---|---|---|---|---|---|---|---|
| | Complete | 214 | | 356/358 | | 431 | |
| | allotype | (allotype) | | (allotype) | | (allotype) | |
| Rituximab | G1m17,1 | K | 17 | D/L | 1 | A | — |
| Veltuzumab | G1m3 | R | 3 | E/M | — | A | — |

In order to reduce the immunogenicity of therapeutic antibodies in individuals of nG1m1 genotype, it is desirable to select the allotype of the antibody to correspond to the G1m3 allotype, characterized by arginine at Kabat 214, and the nG1m1,2 null-allotype, characterized by glutamic acid at Kabat position 356, methionine at Kabat position 358 and alanine at Kabat position 431. Surprisingly, it was found that repeated subcutaneous administration of G1m3 antibodies over a long period of time did not result in a significant immune response. In alternative embodiments, the human IgG4 heavy chain in common with the G1m3 allotype has arginine at Kabat 214, glutamic acid at Kabat 356, methionine at Kabat 359 and alanine at Kabat 431. Since immunogenicity appears to relate at least in part to the residues at those locations, use of the human IgG4 heavy chain constant region sequence for therapeutic antibodies is also a preferred embodiment. Combinations of G1m3 IgG1 antibodies with IgG4 antibodies may also be of use for therapeutic administration.

### Known Antibodies

In various embodiments, the claimed methods and compositions may utilize any of a variety of antibodies known in the art. For example, a gene and its protein product may have been previously reported, but not associated with cancer. Antibodies against the protein product may have been known, but not utilized for cancer therapy. Following identification of a gene as a cancer gene, known antibodies against the gene product may be adapted for use in treating cancer.

Antibodies of potential use may be commercially obtained from a number of known sources. For example, a variety of antibody secreting hybridoma lines are available from the American Type Culture Collection (ATCC, Manassas, Va.). A large number of antibodies against various target antigens have been deposited at the ATCC and/or have published variable region sequences and are available for use in the claimed methods and compositions. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953; 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

Known antibodies may also be used in combination therapy, for example in combination with an siRNA species, a chemotherapeutic agent, a novel antibody against a newly discovered oncogene, radiation therapy, surgery or other known cancer therapeutic modalities. Particular antibodies that may be of use in such combinations include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM5), MN-15 or MN-3 (anti-CEACAM6), Mu-9 (anti-colon-specific antigen-p), Immu 31 (an anti-alpha-fetoprotein), hR1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin) and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,251,164), hAl9 (U.S. Pat. No. 7,109,304), hIMMU-31 (U.S. Pat. No. 7,300,655), hLL1 (U.S. Pat. No. 7,312,318,), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Other useful antigens that may be targeted include carbonic anhydrase IX, alpha-fetoprotein (AFP), α-actinin-4, A3, antigen specific for A33 antibody, ART-4, B7, Ba 733, BAGE, BrE3-antigen, CAl25, CAMEL, CAP-1, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD7OL, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, HIF-1α, colon-specific antigen-p (CSAp), CEACAM5, CEACAM6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-J3, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bc1-2, bc1-6, Kras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

A comprehensive analysis of suitable antigen (Cluster Designation, or CD) targets on hematopoietic malignant cells, as shown by flow cytometry and which can be a guide to selecting suitable antibodies for combination therapy, is Craig and Foon, Blood prepublished online Jan. 15, 2008; DOL 10.1182/blood-2007-11-120535.

The CD66 antigens consist of five different glycoproteins with similar structures, CD66a-e, encoded by the carcinoembryonic antigen (CEA) gene family members, BCG, CGM6, NCA, CGM1 and CEA, respectively. These CD66 antigens (e.g., CEACAM6) are expressed mainly in granulocytes, normal epithelial cells of the digestive tract and tumor cells of various tissues. Also included as suitable targets for cancers are cancer testis antigens, such as NY-ESO-1 (Theurillat et al., Int. J. Cancer 2007; 120(11):2411-7), as well as CD79a in myeloid leukemia (Kozlov et al., Cancer Genet. Cytogenet. 2005; 163(1):62-7) and also B-cell diseases, and CD79b for non-Hodgkin's lymphoma (Poison et al., Blood 110(2):616-623). A number of the aforementioned antigens are disclosed in U.S. Provisional Application Ser. No. 60/426,379, entitled “Use of Multi-specific, Non-covalent Complexes for Targeted Delivery of Therapeutics,” filed Nov. 15, 2002. Cancer stem cells, which are ascribed to be more therapy-resistant precursor malignant cell populations (Hill and Penis, J. Natl. Cancer Inst. 2007; 99:1435-40), have antigens that can be targeted in certain cancer types, such as CD133 in prostate cancer (Maitland et al., Ernst Schering Found. Sympos. Proc. 2006; 5:155-79), non-small-cell lung cancer (Donnenberg et al., J. Control Release 2007; 122(3):385-91), and glioblastoma (Beier et al., Cancer Res. 2007; 67(9):4010-5), and CD44 in colorectal cancer (Dalerba er al., Proc. Natl. Acad. Sci. USA 2007; 104(24)10158-63), pancreatic cancer (Li et al., Cancer Res. 2007; 67(3):1030-7), and in head and neck squamous cell carcinoma (Prince et al., Proc. Natl. Acad. Sci. USA 2007; 104(3)973-8). Another useful target for breast cancer therapy is the LIV-1 antigen described by Taylor et al. (Biochem. J. 2003; 375:51-9).

For multiple myeloma therapy, suitable targeting antibodies have been described against, for example, CD38 and CD138 (Stevenson, Mol Med 2006; 12(11-12):345-346; Tassone et al., Blood 2004; 104(12):3688-96), CD74 (Stein et al., ibid.), CS1 (Tai et al., Blood 2008; 112(4):1329-37, and CD40 (Tai et al., 2005; Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003, J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004, BMC Cancer 12:34; Shachar & Haran, 2011, Leuk Lymphoma 52:1446-54). Milatuzumab (hLL1) is an exemplary anti-CD74 antibody of therapeutic use for treatment of MIF-mediated diseases.

Anti-TNF-α antibodies are known in the art and may be of use to treat cancer. Known antibodies against TNF-α include the human antibody CDP571 (Ofei et al., 2011, Diabetes 45:881-85); murine antibodies MTNFAI, M2TNFAI, M3TNFAI, M3TNFABI, M302B and M303 (Thermo Scientific, Rockford, Ill.); infliximab (Centocor, Malvern, Pa.); certolizumab pegol (UCB, Brussels, Belgium); and adalimumab (Abbott, Abbott Park, Ill.). These and many other known anti-TNF-α antibodies may be used in the claimed methods and compositions.

Checkpoint inhibitor antibodies have been used primarily in cancer therapy. Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response against tumor tissues. Exemplary checkpoint inhibitor antibodies against cytotoxic T-lymphocyte antigen 4 (CTLA4, also known as CD152), programmed cell death protein 1 (PD1, also known as CD279) and programmed cell death 1 ligand 1 (PD-L1, also known as CD274), may be used in combination with one or more other agents to enhance the effectiveness of immune response against cancer cells or tissues. Exemplary anti-PD1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.). Anti-PD1 antibodies are commercially available, for example from ABCAM® (AB137132), BIOLEGEND® (EH12.2H7, RMP1-14) and AFFYMETRIX® EBIOSCIENCE (J105, J116, MIH4). Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX® EBIOSCIENCE (MIH1). Exemplary anti-CTLA4 antibodies include ipilimumab (Bristol-Myers Squibb) and tremelimumab (PFIZER). Anti-PD1 antibodies are commercially available, for example from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89).

In another preferred embodiment, antibodies are used that internalize rapidly and are then re-expressed, processed and presented on cell surfaces, enabling continual uptake and accretion of circulating conjugate by the cell. An example of a most-preferred antibody/antigen pair is LL1, an anti-CD74 MAb (invariant chain, class II-specific chaperone, Ii) (see, e.g., U.S. Pat. Nos. 6,653,104; 7,312,318; the Examples section of each incorporated herein by reference). The CD74 antigen is highly expressed on B-cell lymphomas (including multiple myeloma) and leukemias, certain T-cell lymphomas, melanomas, colonic, lung, and renal cancers, glioblastomas, and certain other cancers (Ong et al., Immunology 98:296-302 (1999)). A review of the use of CD74 antibodies in cancer is contained in Stein et al., Clin Cancer Res. 2007 Sep. 15; 13(18 Pt 2):5556s-5563s, incorporated herein by reference.

The diseases that are preferably treated with anti-CD74 antibodies include, but are not limited to, non-Hodgkin's lymphoma, Hodgkin's disease, melanoma, lung, renal, colonic cancers, glioblastome multiforme, histiocytomas, myeloid leukemias, and multiple myeloma. Continual expression of the CD74 antigen for short periods of time on the surface of target cells, followed by internalization of the antigen, and re-expression of the antigen, enables the targeting LL1 antibody to be internalized along with any chemotherapeutic moiety it carries. This allows a high, and therapeutic, concentration of LL1-chemotherapeutic drug conjugate to be accumulated inside such cells. Internalized LL1-chemotherapeutic drug conjugates are cycled through lysosomes and endosomes, and the chemotherapeutic moiety is released in an active form within the target cells.

### Bispecific and Multispecific Antibodies

Bispecific antibodies are useful in a number of biomedical applications. For instance, a bispecific antibody with binding sites for a tumor cell surface antigen and for a T-cell surface receptor can direct the lysis of specific tumor cells by T cells. Bispecific antibodies recognizing gliomas and the CD3 epitope on T cells have been successfully used in treating brain tumors in human patients (Nitta, et al. Lancet. 1990; 355:368-371). In certain embodiments, the techniques and compositions for therapeutic agent conjugation disclosed herein may be used with bispecific or multispecific antibodies either in combination therapy or for delivery of targeted anti-cancer therapeutic agents, such as siRNA against a newly discovered cancer gene.

Numerous methods to produce bispecific or multispecific antibodies are known, as disclosed, for example, in U.S. Pat. No. 7,405,320, the Examples section of which is incorporated herein by reference. Bispecific antibodies can be produced by the quadroma method, which involves the fusion of two different hybridomas, each producing a monoclonal antibody recognizing a different antigenic site (Milstein and Cuello, Nature, 1983; 305:537-540).

Another method for producing bispecific antibodies uses heterobifunctional cross-linkers to chemically tether two different monoclonal antibodies (Staerz, et al. Nature. 1985; 314:628-631; Perez, et al. Nature. 1985; 316:354-356). Bispecific antibodies can also be produced by reduction of each of two parental monoclonal antibodies to the respective half molecules, which are then mixed and allowed to reoxidize to obtain the hybrid structure (Staerz and Bevan. Proc Natl Acad Sci USA. 1986; 83:1453-1457). Another alternative involves chemically cross-linking two or three separately purified Fab′ fragments using appropriate linkers. (See, e.g., European Patent Application 0453082).

Other methods include improving the efficiency of generating hybrid hybridomas by gene transfer of distinct selectable markers via retrovirus-derived shuttle vectors into respective parental hybridomas, which are fused subsequently (DeMonte, et al. Proc Natl Acad Sci USA. 1990, 87:2941-2945); or transfection of a hybridoma cell line with expression plasmids containing the heavy and light chain genes of a different antibody.

Cognate V_{H} and V_{L} domains can be joined with a peptide linker of appropriate composition and length (usually consisting of more than 12 amino acid residues) to form a single-chain Fv (scFv) with binding activity. Methods of manufacturing scFvs are disclosed in U.S. Pat. No. 4,946,778 and U.S. Pat. No. 5,132,405, the Examples section of each of which is incorporated herein by reference. Reduction of the peptide linker length to less than 12 amino acid residues prevents pairing of V_{H} and V_{L} domains on the same chain and forces pairing of V_{H} and V_{L} domains with complementary domains on other chains, resulting in the formation of functional multimers. Polypeptide chains of V_{H} and V_{L} domains that are joined with linkers between 3 and 12 amino acid residues form predominantly dimers (termed diabodies). With linkers between 0 and 2 amino acid residues, trimers (termed triabody) and tetramers (termed tetrabody) are favored, but the exact patterns of oligomerization appear to depend on the composition as well as the orientation of V-domains (V_{H}-linker-V_{L} or V_{L}-linker-V_{H}), in addition to the linker length.

These techniques for producing multispecific or bispecific antibodies exhibit various difficulties in terms of low yield, necessity for purification, low stability or the labor-intensiveness of the technique. More recently, bispecific constructs known as “DOCK-AND-LOCK™” (DNL™) have been used to produce combinations of virtually any desired antibodies, antibody fragments and other effector molecules (see, e.g., U.S. Pat. Nos. 7,550,143; 7,521,056; 7,534,866; 7,527,787 and U.S. Ser. No. 11/925,408, the Examples section of each of which incorporated herein by reference). The technique utilizes complementary protein binding domains, referred to as anchoring domains (AD) and dimerization and docking domains (DDD), which bind to each other and allow the assembly of complex structures, ranging from dimers, trimers, tetramers, quintamers and hexamers. These form stable complexes in high yield without requirement for extensive purification. The technique allows the assembly of monospecific, bispecific or multispecific antibodies.

Such antibodies can be combined as fusion proteins of various forms, such as IgG, Fab, scFv, and the like, as described in U.S. Pat. Nos. 6,083,477; 6,183,744 and 6,962,702 and U.S. Patent Application Publication Nos. 20030124058; 20030219433; 20040001825; 20040202666; 20040219156; 20040219203; 20040235065; 20050002945; 20050014207; 20050025709; 20050079184; 20050169926; 20050175582; 20050249738; 20060014245 and 20060034759, the Examples section of each incorporated herein by reference.

### Pre-Targeting

Bispecific or multispecific antibodies may also be utilized in pre-targeting techniques. Pre-targeting is a multistep process originally developed to resolve the slow blood clearance of directly targeting antibodies, which contributes to undesirable toxicity to normal tissues such as bone marrow. With pre-targeting, an siRNA, radionuclide or other therapeutic agent may be attached to a small delivery molecule (targetable construct) that is cleared within minutes from the blood. A pre-targeting bispecific or multispecific antibody, which has binding sites for the targetable construct as well as a target antigen, is administered first, free antibody is allowed to clear from circulation and then the targetable construct is administered.

Pre-targeting methods are disclosed, for example, in Goodwin et al., U.S. Pat. No. 4,863,713; Goodwin et al., J. Nucl. Med. 29:226, 1988; Hnatowich et al., J. Nucl. Med. 28:1294, 1987; Oehr et al., J. Nucl. Med. 29:728, 1988; Klibanov et al., J. Nucl. Med. 29:1951, 1988; Sinitsyn et al., J. Nucl. Med. 30:66, 1989; Kalofonos et al., J. Nucl. Med. 31:1791, 1990; Schechter et al., Int. J. Cancer 48:167, 1991; Paganelli et al., Cancer Res. 51:5960, 1991; Paganelli et al., Nucl. Med. Commun. 12:211, 1991; U.S. Pat. No. 5,256,395; Stickney et al., Cancer Res. 51:6650, 1991; Yuan et al., Cancer Res. 51:3119, 1991; U.S. Pat. Nos. 6,077,499; 7,011,812; 7,300,644; 7,074,405; 6,962,702; 7,387,772; 7,052,872; 7,138,103; 6,090,381; 6,472,511; 6,962,702; and 6,962,702, each incorporated herein by reference.

A pre-targeting method of treating or diagnosing a disease or disorder in a subject may be provided by: (1) administering to the subject a bispecific antibody or antibody fragment; (2) optionally administering to the subject a clearing composition, and allowing the composition to clear the antibody from circulation; and (3) administering to the subject the targetable construct, containing one or more chelated or chemically bound therapeutic or diagnostic agents.

### Targetable Constructs

In certain embodiments, targetable construct peptides labeled with one or more therapeutic or diagnostic agents for use in pre-targeting may be selected to bind to a bispecific antibody with one or more binding sites for a targetable construct peptide and one or more binding sites for a tumor-associated antigen. Bispecific antibodies may be used in a pretargeting technique wherein the antibody may be administered first to a subject. Sufficient time may be allowed for the bispecific antibody to bind to a target antigen and for unbound antibody to clear from circulation. Then a targetable construct, such as a labeled peptide, may be administered to the subject and allowed to bind to the bispecific antibody and localize at the diseased cell or tissue.

Such targetable constructs can be of diverse structure and are selected not only for the availability of an antibody or fragment that binds with high affinity to the targetable construct, but also for rapid in vivo clearance when used within the pre-targeting method and bispecific antibodies (bsAb) or multispecific antibodies. Hydrophobic agents are best at eliciting strong immune responses, whereas hydrophilic agents are preferred for rapid in vivo clearance. Thus, a balance between hydrophobic and hydrophilic character is established. This may be accomplished, in part, by using hydrophilic chelating agents to offset the inherent hydrophobicity of many organic moieties. Also, sub-units of the targetable construct may be chosen which have opposite solution properties, for example, peptides, which contain amino acids, some of which are hydrophobic and some of which are hydrophilic.

Peptides having as few as two amino acid residues, preferably two to ten residues, may be used and may also be coupled to other moieties, such as chelating agents. The linker should be a low molecular weight conjugate, preferably having a molecular weight of less than 50,000 daltons, and advantageously less than about 20,000 daltons, 10,000 daltons or 5,000 daltons. More usually, the targetable construct peptide will have four or more residues and one or more haptens for binding, e.g., to a bispecific antibody. Exemplary haptens may include In-DTPA (indium-diethylene triamine pentaacetic acid) or HSG (histamine succinyl glycine). The targetable construct may also comprise one or more chelating moieties, such as DOTA (1,4,7,10-tetraazacyclododecane 1,4,7,10-tetraacetic acid), NOTA (1,4,7-triaza-cyclononane-1,4,7-triacetic acid), TETA (p-bromoacetamido-benzyl-tetraethylaminetetraacetic acid), NETA ([2-(4,7-biscarboxymethyl[1,4,7]triazacyclononan-1-yl-ethyl]-2-carbonylmethyl-amino]acetic acid) or other known chelating moieties. Chelating moieties may be used, for example, to bind to a therapeutic and or diagnostic radionuclide, paramagnetic ion or contrast agent.

The targetable construct may also comprise unnatural amino acids, e.g., D-amino acids, in the backbone structure to increase the stability of the peptide in vivo. In alternative embodiments, other backbone structures such as those constructed from non-natural amino acids or peptoids may be used.

The peptides used as targetable constructs are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). When the peptides are prepared for later use within the bispecific antibody system, they are advantageously cleaved from the resins to generate the corresponding C-terminal amides, in order to inhibit in vivo carboxypeptidase activity.

Where pretargeting with bispecific antibodies is used, the antibody will contain a first binding site for an antigen produced by or associated with a target tissue and a second binding site for a hapten on the targetable construct. Exemplary haptens include, but are not limited to, HSG and In-DTPA. Antibodies raised to the HSG hapten are known (e.g. 679 antibody) and can be easily incorporated into the appropriate bispecific antibody (see, e.g., U.S. Pat. Nos. 6,962,702; 7,138,103 and 7,300,644, incorporated herein by reference with respect to the Examples sections). However, other haptens and antibodies that bind to them are known in the art and may be used, such as In-DTPA and the 734 antibody (e.g., U.S. Pat. No. 7,534,431, the Examples section incorporated herein by reference).

### DOCK-AND-LOCK™ (DNL™)

In certain embodiments, a bivalent or multivalent antibody is formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264. Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Although the standard DNL™ complex comprises a trimer with two DDD-linked molecules attached to one AD-linked molecule, variations in complex structure allow the formation of dimers, trimers, tetramers, pentamers, hexamers and other multimers. In some embodiments, the DNL™ complex may comprise two or more antibodies, antibody fragments or fusion proteins which bind to the same antigenic determinant or to two or more different antigens. The DNL™ complex may also comprise one or more other effectors, such as proteins, peptides, immunomodulators, cytokines, interleukins, interferons, binding proteins, peptide ligands, carrier proteins, toxins, ribonucleases such as onconase, inhibitory oligonucleotides such as siRNA, antigens or xenoantigens, polymers such as PEG, enzymes, therapeutic agents, hormones, cytotoxic agents, anti-angiogenic agents, pro-apoptotic agents or any other molecule or aggregate. Such complexes and their constituent effector moieties are disclosed, for example, in U.S. Pat. Nos. 7,521,056; 7,527,787; 7,534,866; 7,550,143; 7,666,400; 7,858,070; 7,871,622; 7,901,680; 7,906,118; 7,906,121; 7,981,398; 8,003,111; 8,034,352; 8,158,129; 8,163,291; 8,211,440; 8,246,960; 8,277,817; 8,282,934; 8,349,332; 8,435,540; 8,475,794; 8,481,041; 8,491,914; 8,551,480; 8,562,988; 8,597,659; 8,865,176; 8,871,216; 8,883,160; 8,883,162; 8,906,377; the Figures and Examples section of each of which are incorporated herein by reference.

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). Thus, the four isoforms of PKA regulatory subunits are RIα, RIβ, RIIα and RIIβ. The R subunits have been isolated only as stable dimers and the dimerization domain has been shown to consist of the first 44 amino-terminal residues of RIIa (Newlon et al., Nat. Struct. Biol. 1999; 6:222). As discussed below, similar portions of the amino acid sequences of other regulatory subunits are involved in dimerization and docking, each located near the N-terminal end of the regulatory subunit. Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561)

Since the first AKAP, microtubule-associated protein-2, was characterized in 1984 (Lohmann et al., Proc. Natl. Acad. Sci USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human RIIa are both located within the same N-terminal 44 amino acid sequence (Newlon et al., Nat. Struct. Biol. 1999; 6:222; Newlon et al., EMBO J. 2001; 20:1651), which is termed the DDD herein.

We have developed a platform technology to utilize the DDD of human PKA regulatory subunits and the AD of AKAP as an excellent pair of linker modules for docking any two entities, referred to hereafter as A and B, into a noncovalent complex, which could be further locked into a DNL™ complex through the introduction of cysteine residues into both the DDD and AD at strategic positions to facilitate the formation of disulfide bonds. The general methodology of the approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically. Using various combinations of linkers, adaptor modules and precursors, a wide variety of DNL™ constructs of different stoichiometry may be produced and used (see, e.g., U.S. Nos. 7,550,143; 7,521,056; 7,534,866; 7,527,787 and 7,666,400.)

By attaching the DDD and AD away from the functional groups of the two precursors, such site-specific ligations are also expected to preserve the original activities of the two precursors. This approach is modular in nature and potentially can be applied to link, site-specifically and covalently, a wide range of substances, including peptides, proteins, antibodies, antibody fragments, and other effector moieties with a wide range of activities. Utilizing the fusion protein method of constructing AD and DDD conjugated effectors described in the Examples below, virtually any protein or peptide may be incorporated into a DNL™ construct. However, the technique is not limiting and other methods of conjugation may be utilized.

A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, 1989). In such preferred embodiments, the AD and/or DDD moiety may be attached to either the N-terminal or C-terminal end of an effector protein or peptide. However, the skilled artisan will realize that the site of attachment of an AD or DDD moiety to an effector moiety may vary, depending on the chemical nature of the effector moiety and the part(s) of the effector moiety involved in its physiological activity. Site-specific attachment of a variety of effector moieties may be performed using techniques known in the art, such as the use of bivalent cross-linking reagents and/or other chemical conjugation techniques.

For different types of DNL constructs, different AD or DDD sequences may be utilized. Exemplary DDD and AD sequences are provided below.

DDD1
(SEQ ID NO: 74)
SHIQIPPGLTELLQGYTVEVLRQQPPDLVEFAVEYFTRLREARA
DDD2
(SEQ ID NO: 75)
CGHIQIPPGLTELLQGYTVEVLRQQPPDLVEFAVEYFTRLREARA
AD1
(SEQ ID NO: 76)
QIEYLAKQIVDNAIQQA
AD2
(SEQ ID NO: 77)
CGQIEYLAKQIVDNAIQQAGC

The skilled artisan will realize that DDD1 and DDD2 are based on the DDD sequence of the human RIIα isoform of protein kinase A. However, in alternative embodiments, the DDD and AD moieties may be based on the DDD sequence of the human RIα form of protein kinase A and a corresponding AKAP sequence, as exemplified in DDD3, DDD3C and AD3 below.

DDD3
(SEQ ID NO: 78)
SLRECELYVQKHNIQALLKDSIVQLCTARPERPMAFLREYFERLEKEEAK
DDD3C
(SEQ ID NO: 79)
MSCGGSLRECELYVQKHNIQALLKDSIVQLCTARPERPMAFLREYFERLE
KEEAK
AD3
(SEQ ID NO: 80)
CGFEELAWKIAKMIWSDVFQQGC

In other alternative embodiments, other sequence variants of AD and/or DDD moieties may be utilized in construction of the DNL complexes. For example, there are only four variants of human PKA DDD sequences, corresponding to the DDD moieties of PKA RIα, RIIα, RIβ and RIIβ. The RIIα DDD sequence is the basis of DDD1 and DDD2 disclosed above. The four human PKA DDD sequences are shown below. The DDD sequence represents residues 1-44 of RIIα, 1-44 of RIIβ, 12-61 of RIα and 13-66 of RIβ. (Note that the sequence of DDD1 is modified slightly from the human PKA RIIα DDD moiety.)

PKA RIα
(SEQ ID NO: 81)
SLRECELYVQKHNIQALLKDVSIVQLCTARPERPMAFLREYFEKLEKEE
AK
PKA RIβ
(SEQ ID NO: 82)
SLKGCELYVQLHGIQQVLKDCIVHLCISKPERPMKFLREHFEKLEKEENR
QILA
PKA RIIα
(SEQ ID NO: 83)
SHIQIPPGLTELLQGYTVEVGQQPPDLVDFAVEYFTRLREARRQ
PKA RIIβ
(SEQ ID NO: 84)
SIEIPAGLTELLQGFTVEVLRHQPADLLEFALQHFTRLQQENER

### Alternative DNL™ Structures

In certain alternative embodiments, DNL™ constructs may be formed using alternatively constructed antibodies or antibody fragments, in which an AD moiety may be attached at the C-terminal end of the kappa light chain (Cₖ), instead of the C-terminal end of the Fc on the heavy chain. The alternatively formed DNL™ constructs may be prepared as disclosed in Provisional U.S. Patent Application Serial Nos. 61/654,310, filed Jun. 1, 2012, 61/662,086, filed Jun. 20, 2012, 61/673,553, filed Jul. 19, 2012, and 61/682,531, filed Aug. 13, 2012, the entire text of each incorporated herein by reference. The light chain conjugated DNL™ constructs exhibit enhanced Fc-effector function activity in vitro and improved pharmacokinetics, stability and anti-lymphoma activity in vivo (Rossi et al., 2013, Bioconjug Chem 24:63-71).

Cₖ-conjugated DNL™ constructs may be prepared as disclosed in Provisional U.S. Patent Application Serial Nos. 61/654,310, 61/662,086, 61/673,553, and 61/682,531. Briefly, Cₖ-AD2-IgG, was generated by recombinant engineering, whereby the AD2 peptide was fused to the C-terminal end of the kappa light chain. Because the natural C-terminus of C_{K} is a cysteine residue, which forms a disulfide bridge to C_{H}1, a 16-amino acid residue “hinge” linker was used to space the AD2 from the C_{K}-V_{H}1 disulfide bridge. The mammalian expression vectors for Cₖ-AD2-IgG-veltuzumab and Cₖ-AD2-IgG-epratuzumab were constructed using the pdHL2 vector, which was used previously for expression of the homologous C_{H}3-AD2-IgG modules. A 2208-bp nucleotide sequence was synthesized comprising the pdHL2 vector sequence ranging from the Bam HI restriction site within the V_{K}/C_{K} intron to the Xho I restriction site 3′ of the Cₖ intron, with the insertion of the coding sequence for the hinge linker (EFPKPSTPPGSSGGAP, SEQ ID NO:7) and AD2, in frame at the 3′end of the coding sequence for C_{K}. This synthetic sequence was inserted into the IgG-pdHL2 expression vectors for veltuzumab and epratuzumab via Bam HI and Xho I restriction sites. Generation of production clones with SpESFX-10 were performed as described for the C_{H}3-AD2-IgG modules. Cₖ-AD2-IgG-veltuzumab and Cₖ-AD2-IgG-epratuzumab were produced by stably-transfected production clones in batch roller bottle culture, and purified from the supernatant fluid in a single step using MabSelect (GE Healthcare) Protein A affinity chromatography.

Following the same DNL™ process described previously for 22-(20)-(20) (Rossi et al., 2009, Blood 113:6161-71), Cₖ-AD2-IgG-epratuzumab was conjugated with C_{H}1-DDD2-Fab-veltuzumab, a Fab-based module derived from veltuzumab, to generate the bsHexAb 22*-(20)-(20), where the 22* indicates the Cₖ-AD2 module of epratuzumab and each (20) symbolizes a stabilized dimer of veltuzumab Fab. The properties of 22*-(20)-(20) were compared with those of 22-(20)-(20), the homologous Fc-bsHexAb comprising C_{H}3-AD2-IgG-epratuzumab, which has similar composition and molecular size, but a different architecture.

Following the same DNL™ process described previously for 20-2b (Rossi et al., 2009, Blood 114:3864-71), Cₖ-AD2-IgG-veltuzumab, was conjugated with IFNα2b-DDD2, a module of IFNα2b with a DDD2 peptide fused at its C-terminal end, to generate 20*-2b, which comprises veltuzumab with a dimeric IFNα2b fused to each light chain. The properties of 20*-2b were compared with those of 20-2b, which is the homologous Fc-IgG-IFNα.

Each of the bsHexAbs and IgG-IFNα were isolated from the DNL™ reaction mixture by MabSelect affinity chromatography. The two Cₖ-derived prototypes, an anti-CD22/CD20 bispecific hexavalent antibody, comprising epratuzumab (anti-CD22) and four Fabs of veltuzumab (anti-CD20), and a CD20-targeting immunocytokine, comprising veltuzumab and four molecules of interferon-α2b, displayed enhanced Fc-effector functions in vitro, as well as improved pharmacokinetics, stability and anti-lymphoma activity in vivo, compared to their Fc-derived counterparts.

### Phage Display

Certain embodiments of the claimed compositions and/or methods may concern binding peptides and/or peptide mimetics of various target molecules, cells or tissues. Binding peptides may be identified by any method known in the art, including but not limiting to the phage display technique. Various methods of phage display and techniques for producing diverse populations of peptides are well known in the art. For example, U.S. Pat. Nos. 5,223,409; 5,622,699 and 6,068,829 disclose methods for preparing a phage library. The phage display technique involves genetically manipulating bacteriophage so that small peptides can be expressed on their surface (Smith and Scott, 1985, Science 228:1315-1317; Smith and Scott, 1993, Meth. Enzymol. 21:228-257). In addition to peptides, larger protein domains such as single-chain antibodies may also be displayed on the surface of phage particles (Arap et al., 1998, Science 279:377-380).

Targeting amino acid sequences selective for a given organ, tissue, cell type or target molecule may be isolated by panning (Pasqualini and Ruoslahti, 1996, Nature 380:364-366; Pasqualini, 1999, The Quart. J. Nucl. Med. 43:159-162). In brief, a library of phage containing putative targeting peptides is administered to an intact organism or to isolated organs, tissues, cell types or target molecules and samples containing bound phage are collected. Phage that bind to a target may be eluted from a target organ, tissue, cell type or target molecule and then amplified by growing them in host bacteria.

In certain embodiments, the phage may be propagated in host bacteria between rounds of panning Rather than being lysed by the phage, the bacteria may instead secrete multiple copies of phage that display a particular insert. If desired, the amplified phage may be exposed to the target organs, tissues, cell types or target molecule again and collected for additional rounds of panning Multiple rounds of panning may be performed until a population of selective or specific binders is obtained. The amino acid sequence of the peptides may be determined by sequencing the DNA corresponding to the targeting peptide insert in the phage genome. The identified targeting peptide may then be produced as a synthetic peptide by standard protein chemistry techniques (Arap et al., 1998, Smith et al., 1985).

In some embodiments, a subtraction protocol may be used to further reduce background phage binding. The purpose of subtraction is to remove phage from the library that bind to targets other than the target of interest. In alternative embodiments, the phage library may be prescreened against a control cell, tissue or organ. For example, tumor-binding peptides may be identified after prescreening a library against a control normal cell line. After subtraction the library may be screened against the molecule, cell, tissue or organ of interest. Other methods of subtraction protocols are known and may be used in the practice of the claimed methods, for example as disclosed in U.S. Pat. Nos. 5,840,841, 5,705,610, 5,670,312 and 5,492,807.

### Nanobodies

Nanobodies are single-domain antibodies of about 12-15 kDa in size (about 110 amino acids in length). Nanobodies can selectively bind to target antigens, like full-size antibodies, and have similar affinities for antigens. However, because of their much smaller size, they may be capable of better penetration into solid tumors. The smaller size also contributes to the stability of the nanobody, which is more resistant to pH and temperature extremes than full size antibodies (Van Der Linden et al., 1999, Biochim Biophys Act 1431:37-46). Single-domain antibodies were originally developed following the discovery that camelids (camels, alpacas, llamas) possess fully functional antibodies without light chains (e.g., Hamsen et al., 2007, Appl Microbiol Biotechnol 77:13-22). The heavy-chain antibodies consist of a single variable domain (VHH) and two constant domains (C_{H}2 and C_{H}3). Like antibodies, nanobodies may be developed and used as multivalent and/or bispecific constructs. Humanized forms of nanobodies are in commercial development that are targeted to a variety of target antigens, such as IL-6R, vWF, TNF, RSV, RANKL, IL-17A & F and IgE (e.g., ABLYNX®, Ghent, Belgium), with potential clinical use in cancer (e.g., Saerens et al., 2008, Curr Opin Pharmacol 8:600-8; Muyldermans, 2013, Ann Rev Biochem 82:775-97).

The plasma half-life of nanobodies is shorter than that of full-size antibodies, with elimination primarily by the renal route. Because they lack an Fc region, they do not exhibit complement dependent cytotoxicity.

Nanobodies may be produced by immunization of camels, llamas, alpacas or sharks with target antigen, following by isolation of mRNA, cloning into libraries and screening for antigen binding. Nanobody sequences may be humanized by standard techniques (e.g., Jones et al., 1986, Nature 321: 522, Riechmann et al., 1988, Nature 332: 323, Verhoeyen et al., 1988, Science 239: 1534, Carter et al., 1992, Proc. Nat'l Acad. Sci. USA 89: 4285, Sandhu, 1992, Crit. Rev. Biotech. 12: 437, Singer et al., 1993, J. Immun. 150: 2844). Humanization is relatively straight-forward because of the high homology between camelid and human FR sequences. Nanobodies of use are disclosed, for example, in U.S. Pat. Nos. 7,807,162; 7,939,277; 8,188,223; 8,217,140; 8,372,398; 8,557,965; 8,623,361 and 8,629,244, the Examples section of each incorporated herein by reference.)

### Therapeutic Treatment

In another aspect, the invention relates to a method of treating a subject, comprising administering a therapeutically effective amount of a therapeutic agent (e.g., siRNA, antibody, antibody fragment, immunoconjugate) as described herein to a subject. Diseases that may be treated with the therapeutic conjugates described herein include, but are not limited to B-cell malignancies (e.g., non-Hodgkin's lymphoma, mantle cell lymphoma, multiple myeloma, Hodgkin's lymphoma, diffuse large B cell lymphoma, Burkitt lymphoma, follicular lymphoma, acute lymphocytic leukemia, chronic lymphocytic leukemia, hairy cell leukemia). Other diseases include, but are not limited to, adenocarcinomas of endodermally-derived digestive system epithelia, cancers such as breast cancer and non-small cell lung cancer, and other carcinomas, sarcomas, glial tumors, myeloid leukemias, etc. In particular, antibodies against an antigen, e.g., an oncofetal antigen, produced by or associated with a malignant solid tumor or hematopoietic neoplasm, e.g., a gastrointestinal, stomach, colon, esophageal, liver, lung, breast, pancreatic, liver, prostate, ovarian, testicular, brain, bone or lymphatic tumor, a sarcoma or a melanoma, are advantageously used. Such therapeutics can be given once or repeatedly, depending on the disease state and tolerability of the conjugate, and can also be used optionally in combination with other therapeutic modalities, such as surgery, external radiation, radioimmunotherapy, immunotherapy, chemotherapy, antisense therapy, interference RNA therapy, gene therapy, and the like. Each combination will be adapted to the tumor type, stage, patient condition and prior therapy, and other factors considered by the managing physician.

As used herein, the term “subject” refers to any animal (i.e., vertebrates and invertebrates) including, but not limited to mammals, including humans. It is not intended that the term be limited to a particular age or sex. Thus, adult and newborn subjects, as well as fetuses, whether male or female, are encompassed by the term. Doses given herein are for humans, but can be adjusted to the size of other mammals, as well as children, in accordance with weight or square meter size.

Preferably, antibodies used in the treatment of human disease are human or humanized (CDR-grafted) versions of antibodies; although murine and chimeric versions of antibodies can be used. Same species IgG molecules as delivery agents are mostly preferred to minimize immune responses. This is particularly important when considering repeat treatments. For humans, a human or humanized IgG antibody is less likely to generate an anti-IgG immune response from patients. Antibodies such as hLL1 and hLL2 rapidly internalize after binding to internalizing antigen on target cells, which means that a therapeutic agent being carried is rapidly internalized into cells as well. However, antibodies that have slower rates of internalization can also be used to effect selective therapy.

In certain embodiments, a therapeutic agent used for cancer therapy may comprise one or more isotopes. Radioactive isotopes useful for treating diseased tissue include, but are not limited to—¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²²⁷Th and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, I-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

Radionuclides and other metals may be delivered, for example, using chelating groups attached to an antibody or conjugate. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates, such as macrocyclic polyethers for complexing ²²³Ra, may be used.

Therapeutic agents of use may also include, for example, chemotherapeutic drugs such as vinca alkaloids, anthracyclines, epidophyllotoxins, taxanes, antimetabolites, tyrosine kinase inhibitors, alkylating agents, antibiotics, Cox-2 inhibitors, antimitotics, antiangiogenic and proapoptotic agents, particularly doxorubicin, methotrexate, taxol, other camptothecins, and others from these and other classes of anticancer agents, and the like. Other cancer chemotherapeutic drugs include nitrogen mustards, alkyl sulfonates, nitrosoureas, triazenes, folic acid analogs, pyrimidine analogs, purine analogs, platinum coordination complexes, hormones, and the like. Suitable chemotherapeutic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications. Other suitable chemotherapeutic agents, such as experimental drugs, are known to those of skill in the art.

Exemplary drugs of use include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839. Such agents may be used alone or in combination therapy.

Therapeutic agents use for cancer therapy also may comprise toxins conjugated to targeting moieties. Toxins that may be used in this regard include ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin. (See, e.g., Pastan. et al., Cell (1986), 47:641, and Sharkey and Goldenberg, CA Cancer J Clin. 2006 July-August; 56(4):226-43.) Additional toxins suitable for use herein are known to those of skill in the art and are disclosed in U.S. Pat. No. 6,077,499.

Yet another class of therapeutic agent may comprise one or more immunomodulators. Immunomodulators of use may be selected from a cytokine, a stem cell growth factor, a lymphotoxin, an hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β, -γ or -λ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, -γ and -λ; colony stimulating factors (CSFs) such as macrophage-CSF (M-CSF); interleukins (ILs) such as IL-1, IL-1a, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and lymphotoxin (LT). As used herein, the term cytokine includes proteins from natural sources or from recombinant cell culture and biologically active equivalents of the native sequence cytokines.

Chemokines of use include RANTES, MCAF, MIP 1-alpha, MIP 1-Beta and IP-10.

As discussed above, a type of therapeutic agent of particular interest may comprise an inhibitory RNA, such as an siRNA.

Diagnostic Agents

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹⁸F, ⁵²Fe, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III).

Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

### Formulation and Administration

Suitable routes of administration of therapeutic agents include, without limitation, oral, parenteral, subcutaneous, rectal, transmucosal, intestinal administration, intramuscular, intramedullary, intrathecal, direct intraventricular, intravenous, intravitreal, intraperitoneal, intranasal, or intraocular injections. The preferred routes of administration are parenteral. Alternatively, one may administer the compound in a local rather than systemic manner, for example, via injection of the compound directly into a solid tumor.

Therapeutic agents can be formulated according to known methods to prepare pharmaceutically useful compositions, whereby the agent is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

In a preferred embodiment, the therapeutic agent is formulated in Good's biological buffer (pH 6-7), using a buffer selected from the group consisting of N-(2-acetamido)-2-aminoethanesulfonic acid (ACES); N-(2-acetamido)iminodiacetic acid (ADA); N,N-bis(2-hydroxyethyl)-2-aminoethanesulfonic acid (BES); 4-(2-hydroxyethyl)piperazine-1-ethanesulfonic acid (HEPES); 2-(N-morpholino)ethanesulfonic acid (MES); 3-(N-morpholino)propanesulfonic acid (MOPS); 3-(N-morpholinyl)-2-hydroxypropanesulfonic acid (MOPSO); and piperazine-N,N′-bis(2-ethanesulfonic acid) [Pipes]. More preferred buffers are MES or MOPS, preferably in the concentration range of 20 to 100 mM, more preferably about 25 mM. Most preferred is 25 mM MES, pH 6.5. The formulation may further comprise 25 mM trehalose and 0.01% v/v polysorbate 80 as excipients, with the final buffer concentration modified to 22.25 mM as a result of added excipients. The preferred method of storage is as a lyophilized formulation, stored in the temperature range of −20° C. to 2° C., with the most preferred storage at 2° C. to 8° C.

The therapeutic agent can be formulated for intravenous administration via, for example, bolus injection, slow infusion or continuous infusion. Preferably, any antibody of use is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Additional pharmaceutical methods may be employed to control the duration of action of the therapeutic agent. Control release preparations can be prepared through the use of polymers to complex or adsorb the agent. For example, biocompatible polymers include matrices of poly(ethylene-co-vinyl acetate) and matrices of a polyanhydride copolymer of a stearic acid dimer and sebacic acid. Sherwood et al., Bio/Technology 10: 1446 (1992). The rate of release of a therapeutic agent from such a matrix depends upon the molecular weight of the agent, the amount of agent within the matrix, and the size of dispersed particles. Saltzman et al., Biophys. J. 55: 163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Generally, the dosage of an administered therapeutic agent for humans will vary depending upon such factors as the patient's age, weight, height, sex, general medical condition and previous medical history. It may be desirable to provide the recipient with a dosage of, for example, an immunoconjugate that is in the range of from about 1 mg/kg to 24 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy. Preferred dosages may include, but are not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 22 mg/kg and 24 mg/kg. Any amount in the range of 1 to 24 mg/kg may be used. The dosage is preferably administered multiple times, once or twice a week. A minimum dosage schedule of 4 weeks, more preferably 8 weeks, more preferably 16 weeks or longer may be used. The schedule of administration may comprise administration once or twice a week, on a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five week off; (vi) four weeks of therapy followed by one, two, three, four or five week off; (vii) five weeks of therapy followed by one, two, three, four or five week off; and (viii) monthly. The cycle may be repeated 4, 6, 8, 10, 12, 16 or 20 times or more.

Alternatively, an immunoconjugate may be administered as one dosage every 2 or 3 weeks, repeated for a total of at least 3 dosages. Or, twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 12 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

In preferred embodiments, the therapeutic agents are of use for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small cell lung cancer, adenocarcinoma of the lung and squamous carcinoma of the lung, cancer of the peritoneum, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's macroglobulinemia, Wilms' tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. Dysplastic disorders which can be treated include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias; e.g., acute lymphocytic leukemia, acute myelocytic leukemia [including myeloblastic, promyelocytic, myelomonocytic, monocytic, and erythroleukemia]) and chronic leukemias (e.g., chronic myelocytic [granulocytic] leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

### Kits

Various embodiments may concern kits containing components suitable for treating diseased tissue in a patient. Exemplary kits may contain at least one anti-cancer therapeutic agent and/or diagnostic agent as described herein. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### EXAMPLES

The following examples are provided to illustrate, but not to limit, the claims of the present invention.

### Example 1

### Identification of Cancer Genes by In Vivo Fusion of Human Cancer Cells and Animal Cells

After demonstrating, with karyotyping, polymerase chain reaction (PCR) and fluorescence in-situ hybridization, the retention of certain human chromosomes and genes following the spontaneous fusion of human tumor and hamster cells in-vivo, it was postulated that cell fusion causes the horizontal transmission of malignancy and donor genes. Here, we analyzed gene expression profiles of 3 different hybrid tumors first generated in the hamster cheek pouch after human tumor grafting, and then propagated in hamsters and in cell cultures for years: two Hodgkin lymphomas (GW-532, GW-584) and a glioblastoma multiforme (GB-749). Based on the criteria of MAS 5.0 detection P-values <0.065 and at least a 2-fold greater signal expression value than a hamster melanoma control, we identified 3759 probe sets (ranging from 1040 to 1303 in each transplant) from formalin-fixed, paraffin-embedded sections of the 3 hybrid tumors, which unambiguously mapped to 3107 unique Entrez Gene IDs, representative of all human chromosomes; however, by karyology, one of the hybrid tumors (GB-749) had a total of 15 human chromosomes in its cells. Among the genes mapped, 39 probe sets, representing 33 unique Entrez Gene IDs, complied with the detection criteria in all hybrid tumor samples. Five of these 33 genes encode transcription factors that are known to regulate cell growth and differentiation; five encode cell adhesion- and transmigration-associated proteins that participate in oncogenesis and/or metastasis and invasion; and additional genes encode proteins involved in signaling pathways, regulation of apoptosis, DNA repair, and multidrug resistance. These findings were corroborated by PCR and reverse transcription PCR, showing the presence of human alphoid (α)-satellite DNA and the F11R transcripts in additional tumor transplant generations. We posit that in-vivo fusion discloses genes implicated in tumor progression, and gene families coding for the organoid phenotype. Thus, cancer cells can transduce adjacent stromal cells, with the resulting progeny having permanently transcribed genes with malignant and other gene functions of the donor DNA. Using heterospecific in-vivo cell fusion, genes encoding oncogenic and organogenic traits can be identified.

Introduction

The results presented herein indicate that human genes can remain functional within human-hamster hybrid tumors propagated in the animal host, emphasizing the horizontal transmission of human DNA implicated with malignancy and the organoid features of the original patient donor tumors. However, the scope of human DNA transduced and transcribed in these interspecies hybrid cells has not been investigated. Accordingly, we examined (i) if formalin-fixed, paraffin-embedded (FFPE) tumor grafts, which were stored for over 40 years since they were made, could be tested globally for the expression of transcribed human genes, (ii) if human genes are retained during long-term serial passage, and (iii) if there are specific human gene families indigenous to these human-hamster hybrid tumors. By using tumors and hosts of different species, we are able to identify each party's genetic contribution, which is especially problematic when attempting to prove cell-cell fusion in humans, whether involving normal-normal, malignant-normal, or malignant-malignant fusions.

We postulate that these results of heterospecific fusions provide a general mechanism of tumor DNA transfer to stromal cells that results in genetic instability, heterogeneity, and aneuploidy, leading to stable genomic changes associated with cancer progression, while also retaining the tumor's original organoid phenotype, as well as other genes derived from the donor human tumor. This merging of tumor and normal genomes into a new population of malignant hybrid cells could be a mechanism whereby a cancer escapes host immunity by reducing the immunological disparity between the tumor and its host (Goldenberg, 1968, Klin Wochenschr 46:898-99; Goldenberg, 2012, Expert Opin Biol Ther 12(Suppl 1):S133-39).

Materials and Methods

Tumor Xenografts. GW-532

(Fisher et al., 1970, Cancer 25:1286-1300): A male's left axillary Hodgkin lymphoma containing Hodgkin Reed Sternberg (HRS) cells was grafted to the cheek pouches of adult, unconditioned golden hamsters (Mesocricetus auratus), and the resulting tumor was serially passaged in hamsters for >6 years (Goldenberg et al., 2013, PLoS ONE 8:e55324). The transplants were morphologically similar to portions of the original donor specimen, even with HRS cells being identified as early as 17 days after the initial transplantation. This and all subsequent transplant generations showed widespread metastases from the cheek pouch grafts. Transplant generations 2 and 34 were used for genetic analyses.

GW-584

(Goldenberg et al., 2013, PLoS ONE 8:e55324): This was a transplant line established in hamster cheek pouches from the mediastinal Hodgkin lymphoma of a male, also showing HRS cells, and propagated for >5 years. The serial transplants were similar morphologically to the first generation xenograft. The first evidence of metastasis to all major organs and lymph nodes was observed as early as 21 days from the initial grafting, and continued in all subsequent transplant generations, regardless of transplant site. Transplant generation 28 was used for the current studies.

GB-749

(Goldenberg et al., 2012, Int J Cancer 131:49-58): As described earlier (Goldenberg et al., 1974, Nature 250:649-51), this glioblastoma multiforme specimen from an adult female was successfully grafted to the cheek pouch of 1 of 9 unconditioned, adult golden hamsters. This tumor appeared in 14 days and killed the recipient due to widespread metastasis by 4 weeks. This aggressive and rapid growth was continued upon serial passage to other hamsters, showing metastases to all major organs regardless of transplant site in the hamster. Morphologically, the transplant was more uniform and anaplastic than the patient's tumor, but showed the pseudopalisading, lobulated pattern and/or sheets of cells similar to the original patient tumor, even after serial transplantation for >2 years (Goldenberg et al., 1974, Nature 250:649-51; Goldenberg et al., 2012, Int J Cancer 131:49-58). In the original description of this tumor line, karyological studies showed that the malignant cells were heterosynkaryons composed of both human and hamster chromosomes, including 15 human chromosomes (numbers 1, 2, 3, 5, 6, 7, 9, 10, 11, 12, 13, 15, 16,18, and 21, with 6 being identical to the lymphocyte chromosomes of the donor patient) (Goldenberg et al., 2012, Int J Cancer 131:49-58). This was the first experimental evidence of spontaneous in-vivo fusion of human tumor and an animal host's normal cells (Larsson et al., 2008, Histochem Cell Biol 129:551-61), as corroborated by heterosynkaryon formation in the daughter cells.

Recent studies of the GW-532, GW-584, and GB-749 transplants by FISH, RT-PCR, and IHC showed that at least 7 human genes were transcribed in each of these tumor lines, with 3 genes being translated to produce their proteins in the GB-749 line (Goldenberg et al., 2012, Int J Cancer 131:49-58). FISH experiments confirmed the presence of both human and hamster DNA in the same malignant cells in all 3 transplant lines (Goldenberg et al., 2012, Int J Cancer 131:49-58; Goldenberg et al., 2013, PLoS ONE 8:e55324). All FFPE tissues were more than 40 years old, and stored at room temperature.

RNA Samples and Isolation.

FFPE tissues of selected samples (Table 3) were sliced into 4- to 5-μm sections. For each sample, four sections were combined for one total RNA preparation using Qiagen RNEASY® FFPE Kit (Qiagen, Germantown, Md.) according to the manufacturer's instructions. Briefly, the sections were deparaffinized, followed by incubation with proteinase K at 56° C. for 15 min. After inactivation of the proteinase K, the mixture was centrifuged, from which the supernatant was treated with DNase I at room temperature for 15 min, then transferred to a column supplied in the kit. After several washes, the RNA was eluted with 22 μL of RNase-free water. The same procedure was used for preparing total RNA from 4×10⁶ cells of CCL-49, a Syrian golden hamster melanoma cell line purchased from ATCC and cultured in McCoy's 5A medium supplemented with Na-pyruvate, GLUTAMAX™, Penstrep, and 10% FBS.

RNA Quality Control.

Immediately prior to cDNA synthesis, the purity and concentration of RNA samples were determined from OD2601280 readings using a dual beam UV spectrophotometer, and RNA integrity was determined by capillary electrophoresis using the RNA 6000 Nano Lab-on-a-Chip kit and the Bioanalyzer 2100 (Agilent Technologies, Santa Clara, Calif.), as per the manufacturers' instructions.

cDNA Synthesis and Labeling.

RNA (50 ng each sample) was converted to cDNA, amplified by the Single Primer Isothermal Amplifcation (SPIA) method, fragmented and labeled with biotin using OVATION® Pico WTA System v2 and ENCORE® Biotin Module kits according to the manufacturer's instructions (NuGEN, San Carlos Calif.).

Oligonucleotide Array Hybridization and Analysis.

Fragmented, biotinylated cDNA was hybridized for 20 h at 45° C. to GENECHIP® Human U133 X3P Arrays (AFFYMETRIX®, Santa Clara Calif.). The Human U133_X3P arrays contain over 61,000 oligonucleotide probe sets that are specifically designed to interrogate 3′ regions in more than 47,000 different gene transcripts. Arrays were washed and stained with phycoerythrein-conjugated streptavidin (Life Technologies, Carlsbad, Calif.) in a Fluidics Station 450 (AFFYMETRIX®), according to the manufacturer's recommended procedures. Fluorescence intensities were determined using a GCS 3000 7G high-resolution confocal laser scanner, and analyzed using the programs in AGCC and Expression Console (AFFYMETRIX®). MAS 5.0 and RMA Quality Control outputs from Expression Console were used to monitor sample and array performance and identify potential outlier arrays; outlier evaluation was also performed by Principal Components Analysis in GeneMaths XT (Applied Maths, Austin Tex.).

Data Analysis.

Signal expression values and detection P-values were generated by MAS 5.0 (Liu et al., Bioinformatics 18:1593-99; Hubbell et al., 2002, Bioinformatics 18:1585-92; Irizarry et al., 2006, Bioinformatics 22:789-94), after which unannotated probe sets, as well as probe sets with no signal value greater than the median signal for AFFX spike-in controls with all Absent Detection Calls, were omitted from further analysis. Because an intact hamster cell line (CCL-49) control RNA sample was included for comparison with the four human-hamster hybrid FFPE samples, all remaining signal values for the hamster cell line sample were multiplied by the ratio of the median signal in all FFPE hybrid samples for AFFX spike-in control probe sets called present in all samples divided by the median signal for the same probe sets in the hamster CCL-49 sample. Human transcripts were considered positive in human-hamster hybrid FFPE samples if (i) a probe set signal exhibited a 2-fold or greater increase in any FFPE hybrid sample compared to the CCL-49 sample, (ii) the fold change was greater than 2 standard deviations for that probe set across the FFPE samples, and (iii) was called present (P) or marginal (M) for at least one or two FFPE samples (as indicated in the text).

Unsupervised hierarchical clustering and heat map generation were performed in GeneMaths XT (Applied Maths, Belgium) following row mean centering of log 2 transformed MAS 5.0 signal values; probe set and sample clustering were performed by Complete Linkage based on Euclidean distance.

Gene annotation and gene ontology information were obtained from the National Center for Biotechnology Information, NETAFFX™, and the the Gene Ontology Consortium. Pathway annotation and enrichment analysis were performed on-line using WebGestalt (Vanderbilt University). Significant enrichment of specific GO categories or KEGG pathways in each comparison was estimated by hypergeometric tests or chi square tests. Additional bioinformatics analysis was performed using DAVID (Dunham et al., 1992, Hum Genet 88:457-62; Konokpa et al., 2007, J Biol Chem 282:28137-48) and PharmGKB (Klein et al., 2001, Pharmacogenomics J1(3):167-70).

The data files have been deposited in the Gene Expression Omnibus, and can be viewed in the NCBI database at Accession No. GSE58277.

PCR and One-Step Reverse Transcription PCR.

Genomic DNA was isolated from FFPE tissues using QIAAMP® DNA FFPE Tissue Kit (Qiagen, Germantown, Md.) and from Raji or hamster CCL-49 cells using DNEASY® Tissue Kit (Qiagen), according to the manufacturer's instructions. Total RNA was isolated from FFPE tissues using FFPE RNA/DNA Purification Plus Kit (Norgen Biotek, Thorold, Ontario, Canada) and from human HepG2 or hamster CCL-49 cells using TRIZOL® Reagent (Life Technologies, Grand Island, N.Y.).

PCR was performed using a pair of primers (forward: CATCACAAAGAAGTTTCTGAGAATGCTTC, SEQ ID NO:1; reverse: TGCATTCAACTCACAGAGTTGAACCTTCC, SEQ ID NO:2) directed to a conserved region of the 171-bp monomer of human a-satellite DNA (Zhang et al., 2005, Nucl Acids Res 33:W741-48) under the following conditions: 94° C./30 sec, 60° C./30 sec, 72° C./30 sec for 45 or 50 cycles. genomic DNA from human Raji or hamster CCL-49 cells served as positive and negative controls, respectively.

One-step reverse transcription PCR was performed to assess the presence of mRNA transcripts of the F11R gene using SUPERSCRIPT® III One-Step RT-PCR System (Life Technologies) under the following conditions: one cycle of cDNA synthesis (55° C./30 min) and 50 cycles of PCR (94° C./15 sec, 56° C./30 sec, 68° C./30 sec). The pair of primers (UniSTS database) used were: forward: ACTGGGGTCCTTCCATCTCT (SEQ ID NO:85); reverse: CACAACAAGAGCTCCCATT (SEQ ID NO:86). Total RNA from human HepG2, which is known to express F11R (Wang et al., 2013, Nucl Acids Res 41:W77-83), and hamster CCL-49 cells served as positive and negative controls, respectively.

Results

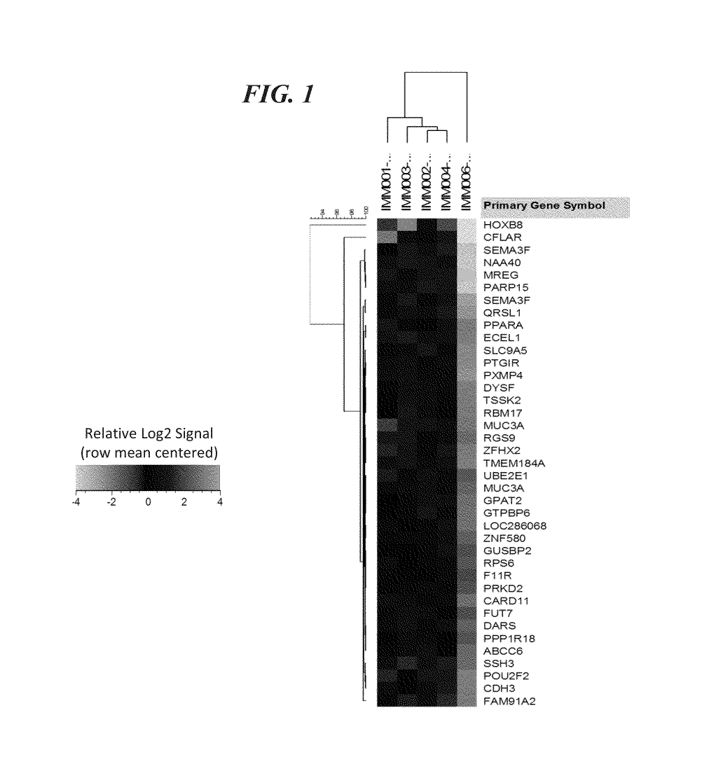

Human mRNA transcripts present in each of four different human-hamster hybrid tumor FFPE samples (Table 3) were identified by analysis of total RNA, in comparison to a control hamster melanoma line (CCL-49), using AFFYMETRIX® Human U133 X3P arrays. Probe sets with MAS 5.0 detection P-values ≦0.065 in a hybrid sample, a detection P-value >0.065 in the hamster control, and an expression signal value that was at least 2-fold greater in the hybrid sample than in the hamster control, were considered to represent expressed human gene transcripts. Using these criteria, we identified a total of 3759 probe sets (ranging from 1040 to 1303 probe sets in at least one hybrid sample), which unambiguously mapped to 3107 unique Entrez Gene IDs (data not shown, see Goldenberg et al., 2014, PLoS ONE 9:e107927, Suppl. Table 1), representing genes from all human chromosomes. Among these, 39 probe sets passed all of the expression criteria in all four hybrid specimens (FIG. 1, Table 4), with 34 probe sets detecting 33 unique Entrez Gene IDs (Table 4), two probe sets detecting either MUC3A or MUC1B, and the remaining probe sets detecting an uncharacterized gene (LOC286068), GUSBP2 or multiple GUSB pseudogenes, and FAM91A2 or multiple uncharacterized genes. Thus, at least 33 unique human genes were transcribed in these FFPE tissues from 3 different human tumor xenografts representing different transplant generations, including two for GW-532, propagated serially for months to years as highly metastatic tumors.

TABLE 3
Characteristics of test articles used in the microarray study.

| RNA sample^{a} | Transplant | Generation | Primary tumor |
|---|---|---|---|
| IMM001 | GW-532 | Gen-2^{b} | Hodgkin lymphoma |
| IMM002 | GW-532 | Gen-34 | Hodgkin lymphoma |
| IMM003 | GW-584 | Gen-28^{c} | Hodgkin lymphoma |
| IMM004 | GB-749 | Gen-4^{d} | Glioma |
| IMM006 | NA^{e} | NA^{e} | Hamster melanoma |

^{a}Prepared from FFPE specimens as indicated, except IMM006, which was prepared from CCL-49, a Syrian golden hamster melanoma cell line acquired from ATCC.
^{b}Human genes of CD74, CXCR4, CD19, CD79b, and VIM were detected by PCR (Ref. 37).
^{c}Human genes of CD74, CXCR4, CD20, and CD79b were detected by PCR (Ref. 37).
^{d}The expression of CD74, CXCR4 and PLAGL2 were detected by IHC staining (Ref. 36).
^{e}Not applicable.

Transcripts of the genes expressed in all four hybrid samples (Table 5) include five encoding transcription factors that are known to regulate cell growth and differentiation (HOXB8, PPARA, POU2F2, ZFHX2, and ZNF580), and five encoding cell adhesion and transmigration-associated proteins that participate in tumorigenesis and/or invasion/metastasis (CDH3, FUT7, F11R, MUC3A, and SEMA3F). In addition, genes whose products are associated with signaling pathways, regulation of apoptosis, DNA repair, and multidrug resistance, also were identified (namely, PRKD2, ECEL1, CARD11, CFLAR, PARP15, and MRP6).

Recognizing that the degraded nature of the FFPE RNA and the high background of hamster RNA in the FFPE hybrid samples could interfere with the sensitivity of MAS 5.0 detection P-values, we relaxed the detection P-value criterion by requiring a detection P-value ≦0.065 in only one of the four hybrid samples, instead of all four, and produced a larger list of human genes that potentially were commonly expressed in all of the hybrid samples. This second list contained 1120 probe sets, representing 982 unique Entrez Gene IDs (data not shown, see Goldenberg et al., 2014, PLoS ONE 9:e107927, Suppl. Table 4). These results indicate the presence of genes for CD20 (MS4A1), CD22, and CD44 (signaling component of the macrophage migration inhibitor factor (MIF)-CD74-CD44 receptor complex), thus corroborating the previous PCR results for the presence of CD20 and, also, CD74 genes in the GW-532 and GW-584 lymphoma hybrid tumors (Goldenberg et al., 2012, Int J Cancer 131:49-58; Goldenberg et al., 2013, PLoS ONE 8:e55324). A number of other human genes, such as those encoding CD24, CD27, CD47, CD52, CD84, CD151, and tenascin XB (TNXB), were found to be transcribed in these hybrid cell lines when the detection P-value criterion was relaxed (data not shown, see Goldenberg et al., 2014, PLoS ONE 9:e107927, Suppl. Table 4).

Pathway enrichment analysis of the larger, relaxed, common gene set and the individual gene sets from each of the four hybrid samples was performed with Webgestalt (Zhang et al., 2005, Nucl Acids Res 33:W741-48; Wang et al., 2013, Nucl. Acids Res 41:W77-83), using the KEGG (Kanehisa et al., 2000, Nucl Acids Res 28:27-30), and Pathway Commons databases (Cerami et al., 2011, Nucl Acids Res 39:D685-90), to identify similar pathways that are commonly represented in all four samples of the three hybrid tumors (data not shown, see Goldenberg et al., 2014, PLoS ONE 9:e107927, Suppl. Table 5). Pathways that were enriched in all five gene sets (the large common gene set and the four individual hybrid sample gene sets) fall into two general categories related to cell-cell communication/focal adhesion/cell junctions/ECM (extracellular matrix) interactions, and cytokine or growth factor signal transduction (including various ErbB signaling pathways). Pathways in two other general categories related to nuclear hormone receptors and MHC antigen processing/presentation were enriched in four of the five gene sets. Enrichment analysis using the DAVID Bioinformatics database (Huang et al., 2009, Nucl Acids Res 37:1-13; Huang et al., 2009, Nature Protoc 4:44-57) identified six functional annotation clusters that were represented in all five gene sets, embryonic morphogenesis, cyclic AMP/adenylate cyclase activity, mitosis/ubiquitin-mediated proteolysis, nuclear hormone receptors, lymphocyte proliferation/activation, and apoptosis (data not shown, see Goldenberg et al., 2014, PLoS ONE 9:e107927, Suppl. Table 6). These results, from both the pathway and functional enrichment analyses, indicate that the various sets of human genes expressed in each hybrid tumor sample affect related cellular processes, and thereby likely produce similar effects on cellular function and growth.

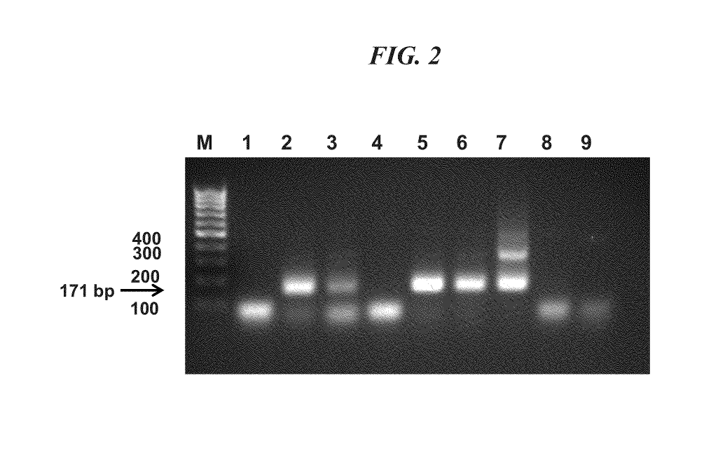

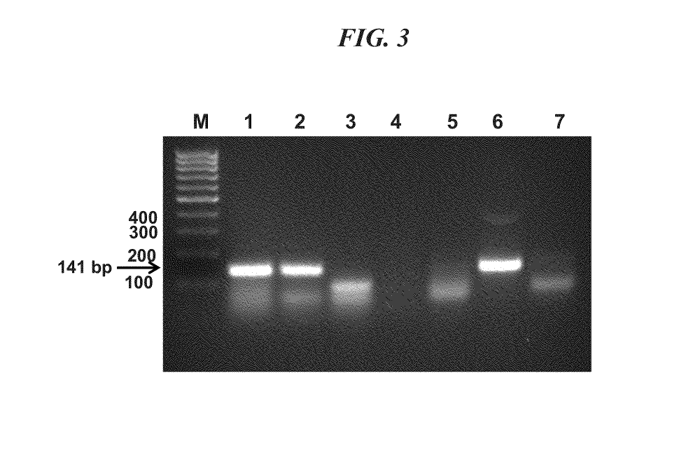

To further corroborate the microarray findings, PCR was performed on six additional FFPE tissue samples: three from GW-532 (generations 11, 52, and 82), one from GW-584 (generation 3), and two from GB-749 (both of generation 2), to assess the presence of human DNA in these tissue blocks, using a pair of primers directed to the 171-bp monomer of human alpha satellite DNA (Dunham et al., 1992, Hum Genet 88:457-62). As shown in FIG. 2, four of the 6 samples (GW-532 generations 52 and 82, GW-584 generation 3, and GB-749 generation 2) were positive for the expected PCR product of human alpha satellite DNA (the 171-bp), which was detected also in the DNA of human lymphoma Raji cells (positive control), but not in the DNA of CCL-49 hamster melanoma cells (negative control). Moreover, we were able to confirm the expression of the F11R gene detected by the cDNA microarray studies in two of the six samples by one-step reverse transcription-PCR, using human hepatic cancer HepG2 cells as the positive control (Konokpa et al., 2007, J Biol Chem 282:28137-48). As shown in FIG. 3, the presence of a 141-bp band was prominent in both GW-532 generation 11 and GW-584 generation 3, as well as in human HepG2 cells (positive control), but not in the tissue of a hamster spleen (negative control). These results were confirmed in a repeat experiment (data not shown), using CCL-49 cells as the negative control.

Discussion

In this Example, we utilized human gene expression microarrays to provide evidence that human genes can remain functional within metastatic human-hamster hybrid tumors propagated in the animal host, and corroborated such findings with additional samples showing the presence of human alphoid (a) satellite DNA and the F11R transcripts by PCR and reverse transcription-PCR, respectively. Our results demonstrate that human tumors transplanted to rodents can merge their DNA with the genome of the animal host, as an example of the larger program of tumor-stromal crosstalk. Cancer cells depend and are influenced by their “soil” or stromal microenvironment (Bhowmick et al., 2004, Nature 432:332-37; Joyce et al., 2009, Nat Rev Cancer 9:239-52; Mueller et al., 2004, Nat Rev Cancer 4:839-49), but it is also known that there can be genetic interchange (Monifer et al., 2000, Cancer Res 60:2562-66; Pelham et al., 2006, Proc Natl Acad Sci USA 103:19848-53). The reciprocal horizontal transfer of genetic material between stromal and tumor cells could explain the heterogeneity and genetic diversity and evolution of cancer cell populations, not only between different patient tumors of the same cancer type, but even different tumors of the same patient, as observed in genetic analyses of human tumor specimens (Burrell et al., 2013, Nature 501:338-45; Stoecklin & Klein, 2012, Int J Cancer 126:589-98; Gerlinger et al., 2012, N Engl J Med 366:883-92). Cell-cell fusion enables the transfer of chromosomes and genetic material from one cell to another, and has been shown to result in viable hybrid progeny capable of replication for different periods, but usually not long-term or as permanent cell lines (Rappa et al., 2012, Am J Pathol 180:2504-15). By using heterospecific cell-cell fusion in-vivo, genes controlling oncogenesis and organoid traits in the donor cancer cells may be elucidated in the fused progeny.

The fusion of tumor and myeloid cells was proposed at the beginning of the 20^{th} century by various German pathologists, such as Aichel, Dor, Hallion, and Kronthal, as cited with the first experimental results and discussion of spontaneous fusion in-vivo in 1968 (Goldenberg, 1968, Klin Wochenschr 46:898-99). This was based on the development of highly aggressive and metastatic tumors after grafting four different human cancers, with one of ovarian cancer origin (GW-127) showing hamster chromosomes, but also retention of human antigens (Goldenberg et al., 1967, Eur J Cancer 3:315-19; Lampert et al., 1968, Arch Geschwulstforsch 32:309-21; Goldenberg et al., 1968, Eur J Cancer 4:547-48; Gotz et al., 1968, Experientia 24:957-58). A series of subsequent studies described the transplantation of diverse human cancers to the cheek pouch of unconditioned (non-immunosuppressed) golden hamsters, and also showed metastases in their hamster hosts as early as 3-4 weeks after grafting, and the presence of both human and hamster markers within the cancer cells. The transplants displayed mostly hamster properties while retaining features of their human origin, including human chromosomes, isoenzyme patterns, antigens, and stathmokinetic properties in response to colchicine that was more compatible with human than hamster cells (Gotz et al., 1968, Experientia 24:957-58; Goldenberg, 1971, Exp Mol Pathol 14:134-37; Goldenberg et al., 1971, Cancer Res 31:1148-52; Goldenberg et al., 1974, Nature 250:649-51). Over the course of about 15 years, while grafting more than 1200 primary human cancers to hamsters (cheek pouch site) or nude mice (subcutaneous site), 15 (1.25%) highly aggressive and metastatic tumors resulted from the hamster transplants (Goldenberg, 2012, Expert Opin Biol Ther 12(Suppl.1):S133-39). These were derived from diverse solid and hematopoietic human tumors, and could be propagated in-vitro or in-vivo for years as permanent cell lines, showing rapid growth and metastatic features typical of a hamster tumor (Fisher et al., 1970, Cancer 25:1286-1300; Goldenberg et al., 1974, Nature 250:649-51; Goldenberg, 2012, Expert Opin Biol Ther 12(Suppl.1):S133-39).

Since gene probes were not available then, it was only recently that FFPE tissues from these earlier transplants were subjected to FISH, PCR, and IHC methods to demonstrate the presence of both species' genetic markers and translation of human genes in some of these permanent transplants, even after years in the foreign, animal host (Goldenberg et al., 2012, Int J Cancer 131:49-58; Goldenberg et al., 2013, PLoS One 8:e55324). For example, the glioblastoma multiforme (GW-749) was reported in 1974 to be a human-hamster hybrid tumor based on retention of up to 15 human and many hamster chromosomes in the same malignant cells, as classified by Giemsa staining, even with definite identification of chromosomes karyotyped from the patient's lymphocytes, thus being a heterosynkaryon (Goldenberg et al., 1974, Nature 250:649-51). More recently, the GW-749 xenograft tumor was shown to have retained 7 transcribed human genes (CD74, CXCR4, PLAGL2, GFAP, VIM, TP53, EGFR), of which CD74, CXCR4, and PLAGL2, continued to be translated to their respective proteins that were visualized by IHC, as well as hamster X chromosome and human pancentromeric DNA in the same nuclei by FISH (Goldenberg et al., 2012, Int J Cancer 131:49-58). Surprisingly, these genes are known to have an association with malignancy and, in particular glial tumors, as well as VIM associated with mesenchymal cells. The transplants continued to express features of the original glioma tumor grafted, even after propagation in hamsters for ˜1 year (Goldenberg et al., 2012, Int J Cancer 131:49-58).

Similar analyses were reported recently for two lymphomas grafted to hamsters (Goldenberg et al., 2013, PLoS One 8:e55324), one of which was described in 1970 and shown to resemble its donor human tumor although gaining highly metastatic properties in the hamster (Fisher et al., 1970, Cancer 25:1286-1300). FISH and PCR analyses showed that these two Hodgkin lymphoma-derived hybrid tumors displayed both hamster and human DNA in the same nuclei by FISH, while also retaining the human genes, CD74, CXCR4, CD19, CD20, CD71, CD79b, and VIM. It is noteworthy that the GB-749 glioblastoma hybrid tumor showed retention of glioma-related genes (PLAGL2, GFAP), whereas the lymphoma-derived hybrid tumor retained several B-cell antigen receptor (BCR)-related genes (CD19, CD20, CD71, CD79b). Three human genes, CD74, CXCR4, and VIM, were common to both the glioblastoma and lymphoma transplants. Both vimentin and CXCR4 are mesenchymal markers associated with epithelial-mesenchymal transition (EMT) whose genes were transcribed in all 3 hybrid tumors examined. It was also suggested that the heterosynkaryons of Hodgkin lymphoma with their Hodgkin Reed-Sternberg (HRS) cells retained their B-cell origin (Goldenberg et al., 2013, PLoS One 8:e55324), confirming other evidence for a B-cell origin of this neoplasm (Marafioti et al., 2000, Blood 95:1443-50), and again corroborated herein by gene probe analysis disclosing B-cell genes (CD20, CD22) in these specimens. As described, these tumors were observed within 2 weeks of their first transplantation, and showed evidence of metastasis in the hamster within 3-4 weeks (Fisher et al., 1970, Cancer 25:1286-1300; Goldenberg et al., 2013, PLoS One 8:e55324), suggesting that the hamster host's early response to the foreign tissue graft may have contributed to this process. Indeed, inflammation and wound healing are known to facilitate cell fusion (Davies et al., 2008, Nat Cell Biot 10:503-5).

In the current Example, we surveyed the extent by which human DNA could be transferred and continuously transcribed in the hybrid tumors. Gene expression microarray analysis was performed using total RNA isolated from FFPE sections of these hybrid tumors, including two different transplant generations of GW-532. Unexpectedly, we detected a combined total of >3000 human genes amongst all of the samples, representing genes from all 23 pairs of human chromosomes, and found that 33 human genes were ubiquitously expressed in each of the 4 samples from the 3 tumors. Five of these genes encode transcription factors that are known to regulate cell growth and differentiation (HOXB8, PPARA, POU2F2, ZFH2, ZNF580), while another five encode cell adhesion and transmigration-associated proteins that are known to participate in tumorigenesis and/or metastatic invasion (CDH3, FUT7, F11R, MUC3A, and SEMA3F). Additional genes whose products can promote metastatic growth were also identified, including two signaling pathway enzymes (PRKD2 and ECEL1), two apoptosis regulators (CARD11 and CFLAR), the DNA repair and apoptosis regulator (PARP1.5), and the multidrug resistance gene (ABCC6). It is particularly noteworthy that published reports show that deregulated expression of either PPARA or POU2F2 can promote oncogenic growth, the developmental function of POU2F2 and HOX genes is to maintain cells in a less-differentiated state (Salmanidis et al., Cell Death Differ 20:1370-80; Vider et al., 1997, Biochem Biophys Res Commun 232:742-8; Pyper et al., 2010, Nucl Recept Signal 8:e002; Youssef et al., 2011, Br J Pharmacol 164:68-82), and high expression of ECEL1 gene was reported by Kawamoto et al. (2003, Int J Oncol 22:815-22) to associate with favorable prognosis in human neuroblastoma. A limitation of this evaluation, however, is the fidelity of the RNA extracted from these FFPE tissues, which were over 40 years old, emphasizing that only positive microarray results can be considered informative. This could explain why some of the genes identified in these specimens by PCR (Goldenberg et al., 2012, Int J Cancer 131:49-58) were not identified by microarray analysis. In this study, however, both the DNA arrays and PCR identified the retention of transcribed human F11R, which codes for a junctional adhesion molecule. The other human gene detected by RT-PCR, α-satellite DNA, is present in the centromere of all human chromosomes, comprising the main structural component of heterochromatin. We should also note that the FFPE sections are of various transplant generations made over many years, and at various times studied in vitro. The populations are very uniform, not reflecting different cell populations morphologically. When the GB-749 glioma transplant was studied after transplantation, several generations showed the presence, in single cells, of both human and hamster chromosomes based on chromosome banding, and in fact compared to chromosomes identified in the donor patient's leukocytes. Since these were in single cells, we referred to these as heterosynkaryons. As such tumors were propagated for long periods, the cell population became very uniform, and there was never evidence of purely human tumor cells being propagated and maintained in serial passage.

Recently, the fusion of human bone marrow stromal cells with two human breast cancer cell lines indicated that the hybrid progeny were more metastatic than the parental breast cancers, and that analysis of coding single-nucleotide polymorphisms by RNA sequencing revealed genetic contributions from both parental partners, with between 1239 and 5345 genes from the parental cells retained in the fused cells (Rappa et al., 2012, Am J Pathol 180:2504-15). However, these fused cells did not show long-term stability, but did retain breast cancer morphology (Rappa et al., 2012, Am J Pathol 180:2504-15). In contrast, fusion of human cancer cells with normal stromal cells of murine mammary glands resulted in malignant tumors that had a sarcomatous appearance (Jacobsen et al., 2006, Cancer Res 66:8274-79). Two different human breast cancer cell populations injected into mice resulted in malignant cells that showed evidence of fusion in the mouse bone marrow, and were more extensively metastatic than the parental cell lines (Mukhopadhyay et al., 2011, PLoS ONE 6:e20473). Similarly, two separate sets of genes that promote metastasis to bone and lung were combined via fusion of breast cancer cell lines, resulting in stable hybrids propagated long-term in cell culture and in-vivo (Lu et al., 2009, Proc Natl Acad Sci USA 106:9385-90). Further, fusion of hematopoietic cells with human and murine epithelial ovarian cancer cells resulted in aggressive tumors of an epithelial phenotype retaining hematopoietic markers (Ramakrishnan et al., 2013, Cancer Res 73:5360-70). It is interesting that the chemokine receptor, CXCR4, which is a promigration marker, was expressed in the hybrid tumors, similar to our own experience of this chemokine's gene being transcribed in the three hybrid tumors studied here.

In our own experiments, the transcribed genes are known to be implicated in tumor progression to invasion and metastasis, including those involving EMT that is postulated to advance tumor cells to more malignant features (Kalluri et al., 2009, J Clin Invest 119:1420-28; Thiery, 2002, Nat Rev Cancer 2:442-54). Recently, in fact, fusions of human lung cancer cells from cell lines and human bone marrow-derived mesenchymal stem cells, when co-cultured in-vitro, showed evidence of cell fusion and the convergence to a mesenchymal-like progeny with EMT and stem cell-like properties, even after injection into NOD/SCID mice (Xu et al., 2014, PLoS ONE 9:e87893). Unfortunately, although considered by these authors as ‘spontaneous’ cell fusion, it is hardly spontaneous when 2 cell lines are grown together in culture, in contrast to growth of tumors that fuse in-vivo with unselected cells in their microenvironment. Nevertheless, such observations provide experimental evidence that in circumstances promoting horizontal gene transfer, whether or not truly spontaneous or the result of experimental conditions, new hybrid daughter tumor cells with new properties are generated, with features of more advanced malignancy (Parris et al., 2013, Crit Rev Oncogen 18:19-42; Pawelek et al., 2008, Nat Rev Cancer 8:377-86; Lu et al., 2009, Proc Natl Acad Sci USA 106:9385-90; Jacobsen et al., 2006, Cancer Res 66:8274-79; Mukhopadhyay et al., 2011, PLoS ONE 6:e20473). Other experiments also have indicated that the progeny hybrid cells after fusion can acquire different properties than the parental cells (Berndt et al., Crit Rev Oncog 18:97-113; Harkness et al., 2013, Crit Rev Oncogen 18:43-74; Lu et al., 2009, Proc Natl Acad Sci USA 106:9385-90; Jacobsen et al., 2006, Cancer Res 66:8274-79; Mukhopadhyay et al., 2011, PLoS ONE 6:e20473); Powell et al., 2011, Cancer Res 71:1497-1505). Thus, such fusion experiments may help further define genes and gene families participating in the evolution, change, and progression of human cancers by methods that are difficult to apply to humans or human tumor specimens directly.

It is intriguing that so many human genes, representing all individual human chromosomes, were transduced, transcribed, and retained permanently in our human-hamster hybrid tumors propagated in-vivo and in-vitro. Despite only 15 human chromosomes being identified by chromosome banding in various cells of two (5^{th} and 15^{th}) transplant generations, which had the full complement of hamster as well as new marker chromosomes, of the GB-749 hybrid tumor derived from the human glioblastoma multiforme (Goldenberg et al., 2012, Int J Cancer 131:49-58), the DNA array results indicate that a total of more than 3000 human genes were detected in a fourth generation passage in hamsters. This discrepancy provokes the speculation that human chromosomal fragments or genes could have translocated to hamster chromosomes, not unlike the DNA sequences (transposable, or “controlling elements”) described by McClintock in maize to relocate to other chromosomes in the genome (McClintock, 1984, Science 226:792-801), and known to regulate the expression of nearby genes. Over the ensuing 60 years, transposable elements, incorrectly referred to previously as ‘junk DNA,” have been confirmed to function in many animal species, including humans (Konkel et al., 2010, Semin Cancer Biol 20:211-221), even the insertion of a transposable element in the human genome that causes hemophilia A (Kazazian et al., 1988, Nature 332:164-66). Retrotransposons (RNA transposons), or McClintock's “jumping genes,” may explain the retention of more human genes in these hybrid tumors than can be accounted for by the 15 human chromosomes identified by chromosome banding, and raises the question of whether similar events result generally with cell-cell fusions between tumor and normal stromal cells. These transposable elements are now understood to alter gene expression and promote genome evolution (Gogvadze et al., 2009, Cell Mo life Sci 66:3727-42). Indeed, lateral gene transfer can occur between microbes and animals (Robinson et al., 2013, PLoS Genetics 9:e1003877), while retrotransposons jumping through the human genome can contribute to oncogenesis (Konkel et al., 2010, Semin Cancer Biol 20:211-221).

In order to reproduce this heterospecific hybridization experimentally, a murine melanoma was fused with hamster cheek pouch fibroblasts in-vitro, and the chromosomes of the daughter cells and their behavior in-vivo in hamsters and genetically-compatible mice were studied (Goldenberg et al., 1975, Int J Cancer 15:282-300). It was found that the murine-hamster hybrid tumor cells (confirmed karyologically) were more malignant in the hamster than the original murine melanoma was in mice, and that the hybrid tumor cells could not be propagated in genetically-compatible mice. Since the original murine melanoma could not grow in adult golden hamsters, the hamster genome came to dominate the genome of the hybrid tumor derived from the murine melanoma, retaining malignancy and metastasizability in hamsters but not in mice, while also losing expression of the melanin present in the original murine melanoma (Goldenberg et al., 1975, Int J Cancer 15:282-300). Evidently, the genetic contribution of the normal (fibroblast) cells governed the biological behavior and genetic features of the hybrid progeny, with the exception of malignancy and metastasizability derived from the murine melanoma. Similar experimental results of melanoma fusions with macrophages in mice have corroborated these findings, but where melanin was retained in the hybrid cells (Pawelek et al., 2008, Nat Rev Cancer 8:377-86; Chakraborty et al., 2000, Cancer Res 60:2512-19; Chakraborty et al., 2001, Gene 275:103-106). Thus, these various studies provide evidence of tumor progression after human-hamster, human-murine, and hamster-murine cell fusions.

The interpretation and relevance of these findings to human cancer are both challenging and stimulating. Does synkaryon formation and the progression of tumors to metastasizability constitute an isolated biological phenomenon without clinical relevance? Tumor heterogeneity has been a focus of interpretation and discussion since the beginnings of cancer histopathology, when diverse cell types and multinucleated giant cells were identified in the tumor and in its microenvironment. These gross cellular observations were then confirmed by genetic studies indicating a heterogeneity between different cells of the same tumor and between different metastases compared among themselves or to the primary tumor cells (Burrell et al., 2013, Nature 501:338-45; Stoecklin & Klein, 2012, Int J Cancer 126:589-98; Gerlinger et al., 2012, N Engl J Med 366:883-92).

Cell-cell fusion may in fact be one mechanism of a more general process of intercellular DNA transfer. Supernatant from human tumor cell cultures or even cell-free DNA from human tumors or sera from cancer patients have been shown to induce tumors in recipient mice (Garcia-Olmo & Garcia-Olmo, 2013, Crit Rev Oncogen 18:153-61; Garcia-Olmo et al., 2010, Cancer Res 70:560-67; Trejo-Becerril et al., 2012, PLoS ONE 7:e52754). Other studies have suggested lateral transfer of non-cellular gene, RNA, or DNA via membrane-derived vesicles, exosomes, or other shed cell constituents (Bergsmedh et al., 2001, Proc Natl Acad Sci USA 98:6407-11; Holmgren et al., 2002, Vox Sang(Suppl 1):5305-06). However, many of these experiments demonstrating oncogenicity utilized immortalized embryonic murine fibroblasts (NIH-3T3), which are known to be susceptible to transformation (Trejo-Becerril et al., 2012, PLoS ONE 7:e52754). Nevertheless, human mutated gene sequences (e.g., KRAS) associated with the primary human cancers were transferred to the transformed murine fibroblasts by plasma DNA taken from human cancer patients, which then proved to be malignant in genetically-compatible mice (Garcia-Olmo et al., 2010, Cancer Res 70:560-67). Others have reported that circulating breast cancer cells exhibit epithelial and mesenchymal traits, with the latter indicating a more aggressive cell population (Xu et al., 2014, PLoS ONE 9:e87893). The basis of this EMT, which has been discussed in many other models of malignancy (Mukhopadhyay et al., 2011, PLoS ONE 6:e20473; Kalluri et al., 2009, J Clin Invest 119:1420-28; Thiery 2002, Nat Rev Cancer 2:442-454), was not elucidated, but does stimulate questioning whether this could be due to DNA transfer, possibly via carcinoma-mesenchymal cell fusion, as already discussed in lung cancer x mesenchymal stem-cell fusion studies (Xu et al., 2014, PLoS ONE 9:e87893). Indeed, it has been hypothesized that circulating cancer cells in humans express myeloid markers as a result of cell fusion (Clawson et al., 2012, PLoS ONE 7:e41052).

These studies suggest that gene or DNA transfer between cells, forming recombinant gene hybrids, may not require cell-cell fusion and synkaryon formation. In fact, most of the recent studies implicating cell fusion are based on evidence of genetic markers of 2 different parental cells in the putative hybrid cell, in the absence of careful chromosome analyses showing a mixed karyotype in single nuclei. Hence, such experiments do not exclude gene transfer without actual synkaryon formation.

In conclusion, if cell-cell fusion is a basic biological process among many species and certain functions in humans (Dittmar et al., 2011, Cell Fusion in Health and Disease. II: Cell Fusion in Disease, Springer, Dordrecht Heidelbert, 203pp; Parris et al., 2013, Crit Rev Oncogen 18:19-42; Larsson et al., 2008, Histochem Cell Biol 129:551-61), it is not unreasonable to expect that it would play an important role in oncogenesis (Dittmar et al., 2011, Cell Fusion in Health and Disease. II: Cell Fusion in Disease, Springer, Dordrecht Heidelbert, 203pp; Duelli et al., 2007, Nat Rev Cancer 7:968-76; Friedl, 2005, Lancet Oncol 6:916-18; Harkness et al, 2013, Crit Rev Oncogen 18:43-74; Lu et al., 2009, Cancer Res 69:8536-39; Parris et al., 2013, Crit Rev Oncogen 18:19-42; Pawelek et al., 2008, Nat Rev Cancer 8:377-86; Vignery, 2005, Trends Cell Biol 15:188-92; Powell et al., 2011, Cancer Res 71:1497-505), accounting for genetic diversity within a single neoplasm or even between different tumors of the same patient. This would amend the long-held view of the clonal derivation of cancer cell populations (Nowell, 1976, Science 194:23-28), now emphasizing that horizontal gene interactions and cell-cell transfer also influence the development and change in cancer cell populations. But the major challenge is to prove that this mechanism is operative in cancer patients, for which evidence is accumulating in unique settings, such as in bone marrow transplantation transferring human chromosomes and genes to the recipients' tumors (Lazova et al., 2013, PLoS ONE 8:e66731), and fusion of myeloma cells and osteoclasts in bone destruction (Cives et al., Crit Rev Oncogen 18:75-96; Andersen et al., 2007, J Pathol 211:10-17). With the increasing interest in the crosstalk and exchange between cancer and stromal cells, including macrophages and leukocytes (Harkness et al., 2013, Crit Rev Oncogen 18:43-74; Pawelek et al, 2008, Nat Rev Cancer 8:377-86), the potential contribution of cell-cell fusion in the horizontal transfer of malignancy and other genes within a tumor deserves continued attention, and implies that this may be a basic biological process occurring between many different cell types both physiologically and in disease. In fact, there is evidence that novel transcriptomes can develop in hybrids that were not present in the parental cells (Harkness et al., 2013, Crit Rev Oncogen 18:43-74; Lu et al., 2009, Proc Natl Acad Sci USA 106:9385-90; Palermo et al., 2009, FASEB J 23:1431-40; Chakraborty et al., 2001, Cell Growth Differ 12:623-30; Berndt et al., 2013, PLoS ONE 8:e63711).

Since the first evidence suggesting that cell fusion is a mechanism by which cancer cells become more diverse and progress to the advanced state of metastasis (Goldenberg et al., 1974, Nature 250:649-51; Goldenberg, 1983, Klin Wochenschr 46:898-99), numerous experiments involving fusions of tumor x tumor, tumor x normal, and tumor x specific myeloid cells, as cited above and in recent reviews (Dittmar et al., 2011, Cell Fusion in Health and Disease. II: Cell Fusion in Disease, Springer, Dordrecht Heidelbert, 203pp; Lu et al., 2009, Cancer Res 69:8536-39; Parris et al., 2013, Crit Rev Oncogen 18:19-42; Pawelek et al., 2008, Nat Rev Cancer 8:377-86; Vignery, 2005, Trends Cell Biol 15:188-92), have made similar conclusions. However, it should be recognized that although revealing important attributes of cell-cell fusion in the recognition and plasticity of gene interactions and the development of hybrid daughter cells with phenotypic diversity, virtually all of these studies have utilized established cancer cell lines mixed either in-vitro or combined in-vivo, with the inherent limitations of cell line selection that may not be representative of the heterogeneous populations of primary tumors. This is emphasized by a publication that appeared while this article was under revision. It was reported that human pontine tumors obtained at autopsy and grafted orthotopically to immune-deficient mice either directly or via intermediate cell culture were different. Direct transplantation resulted in lethal tumors with murine characteristics, whereby the human tumor cells propagated first in-vitro remained human. Interesting, both populations retained the immunophenotype similar to human pontine glioma (Caretti et al., 2014, Acta Neuropathol 127:897-909).

Finally, upon considering the literature on horizontal gene transfer, a distinction should be made between cell-cell fusion, resulting in nuclear merging of two genomes into a single cell, and the horizontal transfer of extracellular DNA as a basis of transduction. Two sets of gene markers derived from different parental cells in the nuclei of progeny cells do not, by themselves, prove one mechanism or the other. These processes should be distinguished in order to devise potential therapeutic strategies to control the horizontal transfer of DNA between malignant and stromal cells in their microenvironment, or to adapt the process to enhance anticancer immunity.

TABLE 4
The 39 probe sets determined to be positive in all hybrid FFPE
specimens. The 33 unique gene transcripts detected by the 39 probe
sets are highlighted in red. The gene (F11R) selected for one-
step reverse transcription PCR is highlighted in blue.

| | Primary Gene | |
|---|---|---|
| Probe Set ID | Symbol | Chromosomal Location |
| Hs.183274.0.A1_3p_at | HOXB8 | chr17q21.3 |
| g2429159_3p_a_at | CFLAR | chr2q33-q34 |
| Hs2.120250.2.S1_3p_a_at | PARP15 | chr3q21.1 |
| 35666_3p_at | SEMA3F | chr3p21.3 |
| g13376118_3p_at | NAA40 | chr11q13.1 |
| Hs.79741.1.S1_3p_at | MREG | chr2q35 |
| 4871689C_3p_s_at | SEMA3F | chr3p21.3 |
| Hs.210778.1.A1_3p_at | QRSL1 | chr6q21 |
| Hs2.132171.1.S1_3p_x_at | SLC9A5 | chr16q22.1 |
| g4502722_3p_at | CDH3 | chr16q22.1 |
| g5454081_3p_at | RBM17 | chr10p15.1 |
| Hs.241205.0.S1_3p_a_at | PXMP4 | chr20q11.22 |
| Hs.147381.0.A1_3p_at | POU2F2 | chr19q13.2 |
| g8923482_3p_s_at | SSH3 | chr11q13.2 |
| Hs.128691.0.S1_3p_at | ZFHX2 | chr14q11.2 |
| g12652612_3p_at | PPARA | chr22q13.31 |
| Hs.126067.0.A1_3p_at | TMEM184A | chr7p22.3 |
| 1555620_3p_a_at | PTGIR | chr19q13.3 |
| g4758231_3p_x_at | ECEL1 | chr2q37.1 |
| Hs.103978.0.S1_3p_x_at | TSSK2 | chr22q11.21 |
| g6912587_3p_at | GTPBP6 | chrXp22.33; Yp11.32 |
| g4506520_3p_a_at | RGS9 | chr17q24 |
| g4503430_3p_at | DYSF | chr2p13.3 |
| Hs.146084.0.A1_3p_at | GPAT2 | chr2q11.1 |
| g12382772_3p_at | CARD11 | chr7p22 |
| Hs.274260.2.S1_3p_at | ABCC6 | chr16p13.1 |
| g12653688_3p_a_at | DARS | chr2q21.3 |
| g7705880_3p_a_at | ZNF580 | chr19q13.42 |
| Hs.163546.0.A1_3p_x_at | UBE2E1 | chr3p24.2 |
| g12751054_3p_s_at | RPS6 | chr9p21 |
| Hs.325905.0.A1_3p_x_at | FUT7 | chr9q34.3 |
| Hs.101150.0.A1_3p_at | PPP1R18 | chr6p21.3 |
| 241669_3p_x_at | PRKD2 | chr19q13.3 |
| g11065890_3p_a_at | F11R | chr1q21.2-q21.3 |
| 1568609_3p_s_at | FAM91A2 | chr1q21.1 |
| Hs.129782.1.S1_3p_a_at | MUC3A | chr7q22 |
| Hs2.376165.1.S1_3p_at | LOC286068 | chr8q11.21 |
| Hs.129782.0.S1_3p_a_at | MUC3A | chr7q22 |
| g5803174_3p_x_at | GUSBP2 | chr5q13///13.2///chr6p21 |

TABLE 5
Notable transcripts of genes present in all four hybrid samples.

| | | | Refer- |
|---|---|---|---|
| Gene | Protein | Function | ence^{a} |
| HOXB8 | Homeobox B8 | Transcriptional | S1 |
| | | factor | |
| POU2F2 | POU class 2 homeobox 2; | Transcriptional | S2 |
| | Oct-2^{b} | factor | |
| ZFHX2 | zinc finger homeodomain-2 | Transcriptional | S3 |
| | | factor | |
| PPARA | peroxisome proliferator- | Transcriptional | S4 |
| | activated receptor alpha | factor | |
| ZNF580 | Zinc finger protein 580 | Transcriptional | S5 |
| | | factor | |
| CDH3 | P-cadherin | Tumor progression | S6 |
| FUT7 | fucosyltransferase 7 | Metastasis | S7 |
| F11R | Junctional adhesion molecule | Tumor | S8 |
| | (JAM)-A^{b}; JAM-1^{b} | proliferation | |
| MUC3A | Mucin 3A | Cell-migration | S9 |
| | | stimulator | |
| SEMA3F | semaphorin 3F | Tumor-suppressor | S10 |
| PRKD2 | Protein kinase D2 | Metastasis | S11 |
| ECEL1 | Endothelin-converting | Zinc | S12 |
| | enzyme-like 1 | metallopeptidase | |
| CARD11 | caspase recruitment domain | Oncogene | S13 |
| | family, member 11 | | |
| CFLAR | c-FLIP | Apoptosis | S14 |
| | | regulator | |
| PARP15 | poly (ADP-ribose) polymerase | Tumor promoting | S15 |
| | family, member 15; BAL3^{b} | factor | |
| MRP6 | multidrug resistance | Multidrug | S16 |
| | associated protein 6; | resistance | |
| | ABCC6^{b} | | |

^{a}A representative publication of each gene or its expressed protein is provided.
^{b}Alternative designation.

What is claimed is:

1. A method of identifying a cancer gene comprising:
a) producing two or more different hybrid tumors by fusion between human cancer cells and normal non-human mammalian cells; and
b) comparing the gene expression profiles of the two or more different hybrid tumor lines to identify human genes that are commonly expressed in the hybrid tumors;
wherein the human genes that are expressed in the two or more different hybrid tumors are identified as cancer genes.

2. The method of claim 1, wherein the normal non-human mammalian cell is a rodent, murine or hamster cell.

3. The method of claim 2, wherein the normal non-human mammalian cell is a golden hamster cell.

4. The method of claim 3, wherein the normal non-human mammalian cell is a golden hamster cheek pouch cell.

5. The method of claim 1, wherein the hybrid tumors are produced in vitro or in vivo.

6. The method of claim 5, wherein the hybrid tumors are produced in vivo after the human cancer cells are transplanted into hamster cheek pouches.

7. The method of claim 5, wherein the hybrid tumors are produced in vitro using a fusogen.

8. The method of claim 7, wherein the fusogen is selected from the group consisting of lysolecithin and Sendai virus.

9. A method of detecting or diagnosing human cancer comprising:
c) identifying one or more cancer genes according to claim 1; and
d) assaying a cell sample from a human subject for expression of the one or more cancer genes;
wherein expression of the cancer genes in the cell sample is indicative of human cancer.

10. The method of claim 9, wherein the cancer gene is selected from the group consisting of ABCC6, CARD11, CDH3, CFLAR, DARS, DYSF, ECEL1, F11R, FAM91A2, FUT7, GPAT2, GTPBP6, GUSBP2, HOXB8, MREG, MUC3A, NAA40, PARP15, POU2F2, PPARA, PPP1R18, PRKD2, PTGIR, PXMP4, QRSL1, RBM17, RGS9, RPS6, SEMA3F, SLC9A5, SSH3, TMEM184A, TSSK2, UBE2E1, ZFHX2, and ZNF580.

11. The method of claim 9, wherein the cancer gene is selected from the group consisting of CARD11, CDH3, CFLAR, ECEL1, F11R, FUT7, HOXB8, MRP6, MUC3A, PARP15, POU2F2, PPARA, PRKD2, SEMA3F, ZFHX2, and ZNF580.

12. The method of claim 9, wherein expression of the one or more cancer genes is indicative of an unfavorable prognosis.

13. The method of claim 9, wherein expression of the one or more cancer genes is associated with metastatic cancer.

14. The method of claim 9, wherein expression of the one or more cancer genes is associated with an organoid phenotype.

15. A method of treating cancer comprising:
c) identifying one or more cancer genes according to claim 1;
d) identifying the protein product of the cancer gene;
e) identifying an inhibitor of the cancer gene or of the protein product of the cancer gene; and
f) administering the inhibitor to an individual with cancer.

16. The method of claim 15, wherein the inhibitor is an antibody.

17. The method of claim 15, wherein the inhibitor is a ligand of the protein product.

18. The method of claim 15, wherein the inhibitor is an siRNA or RNAi.

19. The method of claim 15, wherein the inhibitor is a drug or toxin.

20. The method of claim 15, wherein the protein product is selected from the group consisting of Homeobox B8, POU class 2 homeobox 2 (Oct-2), zinc finger homeodomain-2, peroxisome proliferator-activated receptor alpha, zinc finger protein 580, P-cadherin, fucosyltransferase 7, Junctional adhesion molecule, mucin 3A, semaphorin 3F, protein kinase D2, endothelin-converting enzyme-like 1, caspase recruitment domain family, member 11, c-FLIP, poly (ADP-ribose) polymerase family member 15 and multidrug resistance associated protein 6.

21. A vaccine comprising:
a) a protein product of a cancer gene according to claim 1, or an antigenic fragment thereof; and
b) a binding molecule for an antigen-presenting cell (APC).

22. The vaccine of claim 21, wherein the binding molecule is an antibody that binds to an antigen expressed by an APC.

23. The vaccine of claim 22, wherein the APC antigen is selected from the group consisting of HLA-DR, CD74, CD209 (DC-SIGN), CD34, CD74, CD205, TLR 2 (toll-like receptor 2), TLR 4, TLR 7, TLR 9, BDCA-2, BDCA-3 and BDCA-4.

24. The vaccine of claim 21, wherein the binding molecule is an anti-CD74 or an anti-HLA-DR antibody.

25. A method of treating cancer comprising:
c) identifying one or more cancer genes according to claim 1;
d) producing an RNAi or siRNA that targets the one or more cancer genes; and
e) administering the RNAi or siRNA to an individual with cancer.

26. The method of claim 25, wherein the cancer gene is selected from the group consisting of ABCC6, CARD11, CDH3, CFLAR, DARS, DYSF, ECEL1, F11R, FAM91A2, FUT7, GPAT2, GTPBP6, GUSBP2, HOXB8, MREG, MUC3A, NAA40, PARP15, POU2F2, PPARA, PPP1R18, PRKD2, PTGIR, PXMP4, QRSL1, RBM17, RGS9, RPS6, SEMA3F, SLC9A5, SSH3, TMEM184A, TSSK2, UBE2E1, ZFHX2, and ZNF580.

27. The method of claim 25, wherein the cancer gene is selected from the group consisting of CARD11, CDH3, CFLAR, ECEL1, F11R, FUT7, HOXB8, MRP6, MUC3A, PARP15, POU2F2, PPARA, PRKD2, SEMA3F, ZFHX2, and ZNF580.
